SEQUENCE LISTING (20 sequences)

SEQ ID NO: 1 - protein, 9 aa, Artificial Sequence
  Synthetic
IAKEPVHGV

SEQ ID NO: 2 - protein, 9 aa, Artificial Sequence
  Synthetic
IAKEPVHGA

SEQ ID NO: 3 - protein, 9 aa, Artificial Sequence
  Synthetic
FAPGNAPAL

SEQ ID NO: 4 - protein, 8 aa, Artificial Sequence
  Synthetic
SIINFEKL

SEQ ID NO: 5 - protein, 9 aa, Artificial Sequence
  Synthetic
FAPGNWPAL

SEQ ID NO: 6 - protein, 9 aa, Artificial Sequence
  Synthetic
FAPGNYPAA

SEQ ID NO: 7 - protein, 9 aa, Artificial Sequence
  Synthetic
FAPGNAPAL

SEQ ID NO: 8 - protein, 9 aa, Artificial Sequence
  Synthetic
FAPGNYPAL

SEQ ID NO: 9 - protein, 9 aa, Artificial Sequence
  Synthetic
ILKEPVHGV

SEQ ID NO: 10 - protein, 9 aa, Artificial Sequence
  Synthetic
ILKEPVHGA

SEQ ID NO: 11 - protein, 9 aa, Artificial Sequence
  Synthetic
WLIGFDFDV

SEQ ID NO: 12 - protein, 11 aa, Artificial Sequence
  Synthetic
SGYNFSLGAAV

SEQ ID NO: 13 - protein, 8 aa, Artificial Sequence
  Synthetic
SSPPMFRV

SEQ ID NO: 14 - protein, 8 aa, Artificial Sequence
  Synthetic
RALEYKNL

SEQ ID NO: 15 - protein, 9 aa, Artificial Sequence
  Synthetic
NLVPMVATV

SEQ ID NO: 16 - protein, 9 aa, Artificial Sequence
  Synthetic
VLEETSVML

SEQ ID NO: 17 - protein, 9 aa, Artificial Sequence
  Synthetic
CLGGLLTMV

SEQ ID NO: 18 - protein, 9 aa, Artificial Sequence
  Synthetic
GLCTLVAML

SEQ ID NO: 19 - protein, 9 aa, Artificial Sequence
  Synthetic
YVLDHLIVV

SEQ ID NO: 20 - protein, 9 aa, Artificial Sequence
  Synthetic
LLDQLIEEV

(19) United States
(12) Patent Application Publication
(10) Pub. No.: US 2020/0339655 A1
(43) Pub. Date: Oct. 29, 2020

(54) METHODS FOR PRODUCING A MHC MULTIMER

(71) Applicant: Academisch Ziekenhuis Leiden H.O.D.N. LUMC, Leiden (NL)

(72) Inventors: Jacques Jacobus NEEFJES, Amsterdam (NL); Huib OVAA, Amsterdam (NL); Malgorzata Anna GARSTKA, Amsterdam (NL); Jolien Johanna LUIMSTRA, Amsterdam (NL)

(21) Appl. No.: 16/759,099

(22) PCT Filed: Oct. 26, 2018
(86) PCT No.: PCT/NL2018/050714

§ 371 Date: Apr. 24, 2020

(30) Foreign Application Priority Data
Oct. 26, 2017 (NL) 2019814

(51) Int. Cl. C07K 14/74; G01N 33/569

(52) U.S. Cl. CPC C07K 14/70539; G01N 33/56977

(57) ABSTRACT

The current invention relates to a fast, flexible and efficient method to generate MHC multimers loaded with a desired peptide, by using temperature-mediated peptide exchange. The method may be used at the same time in parallel for different desired peptides. In the method conditional peptides are used that form stable peptide-MHC complexes at low temperatures, but dissociated when exposed to a defined elevated temperature. The resulting conditional MHC I complexes and multimers can be loaded with peptides of choice.

### BACKGROUND OF THE INVENTION

Immune surveillance is mediated by major histocompatibility class I (MHC I) complexes that bind intracellular peptides for presentation to CD8⁺ T lymphocytes. This ability to distinguish between self and foreign is fundamental to adaptive immunity and failure to do so can result in the development of autoimmune diseases. During life humans are under continuous attack by pathogens, such as viruses. Some of them establish lifelong infections, where the virus persists in a latent state without causing symptoms, but occasionally reactivating. One class of such viruses causing recurrent infections are the herpesviruses¹.

Normally reactivation does not lead to disease, because the infection is rapidly cleared by T cells upon recognition of viral antigens. However, in the context of transplantation, when patients are immunocompromised, reactivation of herpesviruses such as cytomegalovirus (CMV) or Epstein-Barr virus (EBV) can result in serious health threats^{2, 3}. It is therefore important to monitor T cell numbers in transplant recipients to predict if a patient will likely clear the infection or if intervention is needed.

The adaptive immune system can be mobilized to our benefit. Immunotherapy, aimed at either suppressing or enhancing cellular immune responses, has advanced greatly over the last decade. Several immune checkpoint inhibitors, including antibodies against CTLA-4 and PD-1/PD-L1, have been approved for use in the clinic and have shown remarkable responses in the treatment of various cancers, including melanoma, non-small-cell lung cancer and renal-cell cancer⁴⁻⁸.

As a consequence of checkpoint blockade, T cell responses elicited against neoantigens are markedly increased, leading to improved killing of cancer cells^{9, 10}. A combination of therapies directed at immune checkpoints and the information in the cancer mutanome holds great promise in personalized cancer treatment. It is therefore crucial to identify T cell responses against neoantigens and other presented cancer-specific epitopes that contribute to the success of immunotherapy.

Since their first use in 1996 by Altman et al., MHC multimers—oligomers of MHC monomers loaded with antigenic peptides—tagged with fluorochrome(s) have been the most extensively used reagents for the analysis and monitoring of antigen-specific T cells by flow cytometry¹¹.

However, multimer generation involves many time-consuming steps, including expression of, for example, MHC I heavy chain and β2-microglubulin in bacteria, refolding with a desired peptide, purification, biotinylation and multimerization¹¹. Initially, all these steps had to be undertaken for every individual peptide-MHC I complex, since empty MHC I molecules are unstable¹².

This prompted the search for ways to generate peptide-receptive MHC molecules, including MHC I molecules, at will to allow parallel production of multiple MHC multimers from a single input MHC I-peptide complex. For example, several techniques aimed at peptide exchange on MHC I have been developed by us and others, such as using periodate or dithionite as a chemical trigger to cleave conditional ligands in situ, or using dipeptides as catalysts, after which peptide remnants can dissociate to be replaced by a peptide of choice¹³⁻¹⁶.

Alternatively, MHC monomers are refolded with a photocleavable peptide that gets cleaved upon UV exposure, after which individual peptide remnants dissociate and empty MHC I molecules can be loaded with peptides of choice and subsequently multimerized¹⁷⁻¹⁹. This approach has facilitated the discovery of a myriad of epitopes and the monitoring of corresponding T cells^{18, 20-22}. However, UV exchange technology requires the use of a photocleavable peptide and a UV source. UV exposure and ligand exchange are not compatible with fluorescently-labeled multimers and the biotinylated peptide-loaded MHC I molecules need to be multimerized on streptavidin post exchange. Other disadvantages include the generation of reactive nitroso species upon UV-mediated cleavage and photo damage of MHC I and/or associated peptides, while the generated heat causes sample evaporation.

In light of this, further methods, products, compositions, and uses for providing MHC molecules with a desired peptide are desired. In particular fast, flexible and efficient methods to generate MHC multimers loaded with a desired peptide would be highly desirable, but are not yet readily available. In particular there is a clear need in the art for reliable, efficient and reproducible products, compositions, methods and uses that allow to provide such MHC multimers, using peptide exchange. Preferably the method can be performed on MHC multimers directly, and avoid the need for post peptide exchange multimerization of the MHC molecules. Accordingly, the technical problem underlying the present invention can been seen in the provision of such products, compositions, methods and uses for complying with any of the aforementioned needs. The technical problem is solved by the embodiments characterized in the claims and herein below.

### DESCRIPTION

### Drawings

Embodiments of the invention are further described hereinafter with reference to the accompanying drawings, in which:

FIG. 1. Temperature-induced peptide exchange allows for the generation of MHC I complexes with high- and low-affinity peptides. (a) Schematic representation of temperature-induced peptide exchange on MHC I molecules. The thermolabile MHC I-peptide complex is stable at 4° C., but undergoes unfolding and degradation under thermal challenge (upper panel). Addition of a higher affinity peptide stabilizes the MHC I, preventing its degradation (lower panel). (b) Primary data of temperature-induced peptide exchange analyzed by gel filtration chromatography at room temperature. MHC I-unstable peptide complex (H-2K^{b}-FAPGNAPAL) and the exchange peptide (0.5 μM and 50 μM, respectively) were incubated at the indicated temperature over a range of time points. The following exchange peptides were used: optimal binder: SIINFEKL (OVA); suboptimal binders: FAPGNWPAL or FAPGNYPAA. One of three representative experiments is shown. (c) The exchange efficiency was calculated from the area under the curve measured by HPLC and normalized to binding of the optimal peptide SIINFEKL for 1 h. Average values±SD from three independent experiments are shown.

FIG. 2: Temperature-exchanged H-2K^{b} multimers efficiently stain antigen-specific CD8³⁰ T cells. (a) Schematic representation of MHC I peptide exchange on monomers (Exchange first, upper panel) or on multimers (Multimerization first, lower panel). (b) Dot plots of MHC I multimer staining of splenocytes from OT-I mice. Multimers were prepared after or before exchanging the input peptide for either a relevant peptide (SIINFEKL, OVA, upper panel) or an irrelevant peptide (FAPGNYPAL, Sendai virus, lower panel) for 30 min at room temperature. Control multimers were prepared using UV-mediated exchange technology on monomers followed by multimerisation. Representative data from three independent experiments are shown. (c) Thermolabile multimers of H-2K^{b}-FAPGNAPAL are stable over time when stored at −80° C. in the presence of 300 mM NaCl or 10% glycerol. H-2K^{b}-FAPGNAPAL multimers were thawed and FAPGNAPAL was exchanged for SIINFEKL prior to staining OT-I splenocytes.

FIG. 3. Temperature-exchanged H-2K^{b} multimers are suitable for staining antigen-specific T cells from virus-infected mice. (a) Primary data of temperature-induced peptide exchange on H-2K^{b} monomers analyzed by gel filtration chromatography at room temperature. The following peptides were used for exchange: SIINFEKL (OVA), FAPGNAPAL (Sendai virus), SGYNFSLGAAV (LCMV NP238), SSPPMFRV (MCMV M38) or RALEYKNL (MCMV 1E3) for 5 min at 20° C. One of two representative experiments is shown. (b) Exchange efficiency was calculated from the area under the curve from HPLC chromatograms normalized to the binding of optimal peptide (SIINFEKL). Average values±SD from two independent experiments are shown. (c) Peptide exchange was performed on H-2K^{b}-FAPGNAPAL multimers for 5 min at 20° C. and multimers were subsequently used to stain corresponding CD8⁺ T cells from PBMCs of an LCMV-infected mouse or splenocytes from an MCMV-infected mouse. Detected percentages of CD8⁺ T cells were comparable between temperature-exchanged multimers and conventional multimers. Irrelevant peptide: FAPGNYPAL (Sendai virus). One of two representative experiments is shown.

FIG. 4. Temperature-exchanged HLA-A*02:01 multimers are suitable for staining virus-specific T cells. HLA-A*02:01-IAKEPVHGV monomers (a-b) or multimers (c) were exchanged for HCMV pp65-A2/NLVPMVATV, HCMV IE-1-A2/VLEETSVML, EBV BMLF-1-A2/GLCTLVAML, EBV LMP2-A2/CLGGLLTMV, EBV BRLF-1-A2/YVLDHLIVV or HAdV E1A-A2/LLDQLIEEV) for 3 h at 32° C. (a) Exchange on monomers analyzed by gel filtration chromatography at room temperature. (b) Efficiency of exchange calculated from the area under the curve from HPLC chromatograms normalized to the binding in respect to input peptide. Average values±SD from two independent experiments are shown. (c) Exchanged multimers were subsequently used for staining of specific T cell clones or T cell lines. Detected percent-ages of multimer-positive CD8⁺ T cells were comparable between temperature-exchanged multimers and conventional multimers. One of two representative experiments is shown.

FIG. 5. Temperature-exchanged multimers used for monitoring of HCMV- and EBV-specific T cells in peripheral blood of an allogeneic stem cell transplantation recipient. Peripheral blood samples taken after allogeneic stem cell transplantation were analyzed for virus-specific CD8⁺ T cells in relation to viral DNA loads (grey). The frequency of HCMV- and EBV-specific T cells within the CD8⁺ T cell populations was determined using temperature-exchanged (dark colors) and conventional (light colors) MHC I multimer staining analyzed by flow cytometry. Average values±SD from two experiments performed on the same day are shown.

FIG. 6. Defining the temperature range for temperature-induced peptide exchange. Thermal denaturation of MHC class I-peptide complexes measured by bary-centric mean fluorescence (BCM, in black). The fit to the first derivate of BCM (in red) shows the melting temperature, Tm, as a maximum: H-2K^{b}-FAPGNAPAL, HLA-A*02:01-ILKEPVHGV, HLA-A*02:01-ILKEPVHGA, and HLA-A*02:01-IAKEPVHGV, HLA-A*02:01-IAKEPVHGA. One of four representative experiments is shown. Melting temperatures are average values±SD from four independent experiments.

FIG. 7. HLA-A*02:01 in complex with IAKEPVHGV peptide is the most suitable for temperature-induced exchange. HLA-A*02:01-ILKEPVHGV, HLA-A*02:01-ILKEPVHGA, HLA-A*02:01-IAKEPVHGV and HLA-A*02:01-IAKEPVHGA complexes were exchanged for a high affinity peptide (vaccinia virus epitope WLIGFDFDV) at indicated temperatures and times. HLA-A*02:01 was used at a concentration of 0.5 μM and exchange pep-tide was used at a concentration of 50 μM. HLA-A*02:01-ILKEPVHGV and HLA-A*02:01-ILKEPVHGA remain stable at RT, but HLA-A*02:01-IAKEPVHGV and HLA-A*02:01-IAKEPVHGA complexes are unstable at RT and are therefore suitable for exchange.

### DEFINITIONS

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

The singular form terms “A,” “an,” and “the” include plural referents unless the content clearly dictates otherwise. Thus, for example, reference to “a cell” includes a combination of two or more cells, and the like.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, etc. As used herein, the term “at most” a particular value means that particular value or less. For example, “at most 5” is understood to be the same as “5 or less” i.e., 5, 4, 3, −10, −11, etc.

The term “comprising” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.”

“Conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art.

“Exemplary” means “serving as an example, instance, or illustration,” and should not be construed as excluding other configurations disclosed herein.

Common amino acid abbreviations, which may be used throughout the text are:

| | | |
|---|---|---|
| Ala | A | Alanine |
| Arg | R | Arginine |
| Asn | N | Asparagine |
| Asp | D | Aspartic acid (Aspartate) |
| Cys | C | Cysteine |
| Gln | Q | Glutamine |
| Glu | E | Glutamic acid (Glutamate) |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |
| Asx | B | Aspartic acid or Asparagine |
| Glx | Z | Glutamine or Glutamic acid |
| Xaa | X | Any amino acid (sometime — is used to refer to any amino acid). |

As used herein the term “MHC molecule” refers to both MHC monomers and/or multimers, e.g. any oligomeric form of one or more MHC molecules. A multimer as described herein is a multimeric proteinaceous molecule (a multimer) comprising at least two members that bind each other via a region of noncovalent interaction, wherein at least one of said at least two members comprises a (poly) peptide chain. A monomer is used herein to refer to a molecule wherein the building blocks are still covalently associated with each other when all noncovalent bonds are broken. The more than one monomer in the multimer may be the same or different from each other. MHC multimers thus include MHC-dimers, MHC-trimers, MHC-tetramers, MHC-pentamers, MHC-hexamers, MHC-dexamers, as well as organic molecules, cells, membranes, polymers and particles that comprise two or more MHC-peptide complexes.

The major histocompatibility complex (MHC) complexes function as antigenic peptide receptors, collecting peptides inside the cell and transporting them to the cell surface, where the MHC-peptide complex can be recognized by T lymphocytes. The human MHC is also called the HLA (human leukocyte antigen) complex (often just the HLA). The mouse MHC is called the H-2 complex or H-2. MHC play a crucial role in the human immune system and a multitude of strategies has been developed to enhance this natural defense system and boost immunity against pathogens or malignancies. MHC molecules, such as MHC class I molecules, particularly HLA-A molecules are valuable tools to identify and quantify specific T cell populations and evaluate cellular immunity in relation to a disease. HLA-A molecules belong to the MHC class I molecules, and are often referred to as “HLA-A class I” or “HLA-A I” molecules. MHC class I molecules also comprises, beside HLA-A molecules, HLA-B and HLA-C molecules, which also play an important role in the immune system. MHC complexes find use in immune monitoring and may be applied to isolate specific T cells for cellular immunotherapy against pathogens or malignancies. MHC complexes may also be used to selectively eliminate undesired specific T cell populations in T cell-mediated diseases.

Two subtypes of MHC molecules exist, MHC Class I and II molecules. These subtypes correspond to two subsets of T lymphocytes: 1) CD8⁺ cytotoxic T cells, which usually recognize peptides presented by MHC Class I molecules (i.e. peptide bound in the peptide binding groove of the MHC), and kill infected or mutated cells, and 2) CD4⁺ helper T cells, which usually recognize peptides presented by MHC Class II molecules (i.e. peptide bound in the peptide binding groove of the MHC), and regulate the responses of other cells of the immune system.

A variety of relatively invariant MHC Class I molecule like molecules have been identified. This group comprises CD1d, HLA E, HLA G, HLA H, HLA F, MICA, MIC B, ULBP-1, ULBP-2, and ULBP-3. HLA-A, B, C are MHC Class I molecules found in humans while MHC class I molecules in mice are designated H-2K, H-2D and H-2L. HLA-A class I molecules play a central role in the immune system and are expressed on the surface of nearly all nucleated cells. Therefore, HLA-A molecules represent valuable tools, particularly for human research and drug development aimed for humans. More particularly, HLA-A molecules can be advantageously used to identify and quantify specific T cell populations and evaluate cellular immunity in relation to a disease in humans, as HLA-A finds use in immune monitoring and may be applied to isolate specific T cells for cellular immunotherapy against pathogens or malignancies in the context of various diseases or conditions such as cancers. MHC complexes such as HLA-A complexes may also be used to selectively eliminate undesired specific T cell populations in T cell-mediated diseases.

It is acknowledged that, in general, the design of peptides suitable for temperature exchange on HLA (e.g. HLA-A allele such as HLA-A*02:01) is nota trivial task. Specifically, it has been found that finding peptides suitable for temperature exchange on human HLA alleles, particularly HLA-A allele(e.g. HLA-A*02:01), is more challenging than for mouse MHC I alleles such as H-2K^{b}. One reason for this is because of the intrinsically higher stability of human MHC class I complexes compared to murine MHC class I complexes.

Another reason is the differential positioning of the first primary anchor as well as the secondary anchor between human HLA alleles ((e.g. HLA-A allele such as HLA-A*02:01) and mouse MHC I alleles (such as H-2K^{b}). For instance, in human HLA-A, the first primary anchor is located at position 2 of the peptide while in mouse MHC I alleles it is located in the middle of the peptide, although it depends on mouse allele (e.g. the same peptide FAPGNYPAL binds to H-2Kb and H-2Db, but to the first one it binds with Tyr, while to the second one with Asn) (Glithero et al (1999), Immunity, Vol. 10, pages 63-74). As for the position of the secondary anchor, it is located in the middle of the peptide in human HLA-A, while in mouse MHC I, it is usually located at position 3 of the peptide. Furthermore, the secondary anchor is not observed for all peptides.

Depending on the MHC molecule, the domains responsible for binding of the peptide have different nomenclatures. Typically two domains are required for specifically binding a peptide, as exemplified by the alpha1 and alpha2 domains of an MHC class I molecule, which are the functional parts of an MHC molecule involved in binding of a peptide. An MHC molecule typically contains other domains not involved in peptide binding. An example of MHC molecule may be one as described by Garboczi D N et al. (Proc Natl Acad Sci USA. 1992 Apr. 15; 89(8):3429-33.). In a preferred embodiment the MHC molecule is in the form of a multimer, comprising more than one MHC monomer.

For example, the most commonly used MHC multimers are tetramers. These are typically produced by, for example, biotinylation of soluble MHC monomers, which are typically produced recombinantly in eukaryotic or bacterial cells. These monomers then bind to a backbone, such as streptavidin or avidin, creating a tetravalent structure.

Monomer and soluble forms of cognate as well as modified MHC molecules e.g. single chain protein with peptide, heavy and light chains fused into one construct, have been produced in bacteria as well as eukaryotic cells. Such forms are also included under the term “MHC molecule”, as well as those MHC molecules that comprise modification, such as modifications that are not in the peptide binding domains or that are in the variable domains of the peptide binding domains of MHC molecules. These modifications may alter the binding specificity of the MHC molecule (i.e. which peptide is bound).

The term “in vivo” refers to an event that takes place in a subject's body; the term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding that MHC molecules, particularly MHC class I molecules, more particularly HLA-A molecules, both monomers as well as multimers, may be provided with a desired peptide (e.g. antigenic peptide) using a fast, reliable and reproducible method that is devoid of various of the disadvantages of methods known in the art.

In the method, a template peptide, bound to the peptide binding groove of a MHC molecule such as MHC class I, preferably a HLA-A molecule, may be exchanged with a desired peptide (i.e. a peptide that one wants to be displayed by the MHC molecule) by increasing the temperature of the MHC molecule provided with the template peptide, causing dissociation of the template peptide from the MHC molecule, and binding of the desired peptide to the MHC molecule.

The method provided for a fast protocol to obtain, in high yield and with high purity, MHC molecules such as MHC class I, preferably HLA-A molecules, that are loaded with a desired peptide.

An important aspect of the current invention is that the MHC molecule such as MHC class I, preferably a HLA-A molecule, with the template peptide may be provided in the form of a multimer, and that the exchange with the desired peptide may be performed directly using the multimer. With the method of the current invention, and in contrast to the methods of the art, the step of multimerization of monomers loaded with a desired peptide may be abolished.

More in particular, it was surprisingly found that an improved peptide exchange technology for providing MHC molecules such as MHC class I molecules, particularly HLA-A molecules, may be provided by the design of low-affinity peptides with low off-rate at reduced temperature, e.g. below 10 degrees Celsius, e.g. at 4° C., and that in a temperature-dependent manner can be exchanged for exogenous peptides of interest (desired peptide).

In other words, the current invention advantageously uses a template peptide to stabilize MHC molecules such as MHC class I molecules, preferably HLA-A molecules, at a reduced temperature, wherein such MHC molecules with the template peptide can effectively be provided with a desired peptide by dissociating the template peptide at an increased temperature and replacement thereof by the desired peptide. In particular this allows for a reliable, robust and reproducible method wherein, in parallel, MHC molecules, preferably HLA-A molecules, may be loaded with different desired peptides, for example using a 96 well plate system or the like, while using the same MHC molecules loaded with a template peptide in each of the parallel experiment.

Indeed, an obstacle of the effective application of MHC molecules provided with a defined peptide is the difficulty of the production methods in the art. It is well-known that MHC molecules are unstable, in particular for MHC class I molecules, more particularly HLA-A molecules, when no antigen is bound. This thus requires that during the process MHC molecules are produced in which the desired peptide is (already) bound. Using prior art methods, the exchange of this template peptide for a desired peptide is highly inefficient since dissociation of the used template peptides is slow under the conditions used or causes destabilization of the MHC molecule (see also Bakker A H et al. Curr Opin Immunol. 2005 August; 17(4):428-33). A frequently used method for multimer generation is UV-mediated peptide exchange. In this method, MHC monomers are refolded with a photocleavable peptide that gets cleaved upon UV exposure, after which individual peptide remnants dissociate and empty MHC I molecules (e.g. HLA-A molecules) can be loaded with peptides of choice and subsequently multimerized. However, UV exchange technology requires the use of a photocleavable peptide and a UV source. UV exposure and ligand exchange are not compatible with fluorescently-labeled multimers and the biotinylated peptide-loaded MHC I molecules need to be multimerized on streptavidin post exchange.

With the method as disclosed herein, such technological difficulties can be overcome.

Thus, according to the invention there is provided for a method for producing a MHC molecule, the method comprising
a. Providing at a reduced temperature an MHC molecule, preferably a MHC class I molecule, more preferably a HLA-A molecule, having bound thereto in the peptide-binding groove of said MHC molecule a template peptide that dissociates from said MHC molecule at an increased temperature;
b. Changing the temperature to an increased temperature, therewith dissociating the template peptide from said MHC molecule; and
c. Contacting the MHC molecule at said increased temperature with a desired peptide for binding to the peptide-binding groove of said MHC molecule, under conditions allowing the desired peptide to bind to the peptide-binding groove of said MHC molecule.

In the method, in step a) an MHC molecule such as MHC class I, preferably a HLA-A molecule, is provided wherein the peptide-binding groove of the MHC molecule is provided with a template peptide. The loaded template peptide is bound or associated with the MHC molecule via the peptide-binding groove. In step a) the MHC molecule, preferably a HLA-A molecule, having bound thereto in the peptide-binding groove of said MHC molecule the template peptide is provided at a temperature wherein the MHC molecule and the template peptide are stable, in other words wherein the template peptide does not dissociate from the MHC molecule, or does not dissociate from the MHC molecule to such extent that there is a substantial loss (e.g. more than 10%, 20%, 30%, 40%, 50%, 60%, 70% loss of total) of properly folded MHC molecule due to instability of the MHC molecule. Such temperature may be referred to herein as a “reduced temperature”. In contrast, an “increased temperature”, as referred to herein, denotes a temperature at which the template peptide dissociates from the MHC molecule. In the absence of any desired peptide (i.e. a desired ligand for the MHC molecule) that is capable of association with the MHC molecule, at the increased temperature, this will cause the MHC molecule to become unstable at the increased temperature, leading to MHC molecule that is not properly folded anymore. It may cause unfolding and precipitation of the MHC molecule.

In a preferred embodiment, the MHC molecule of step a) is a HLA-A molecule (any suitable HLA-A molecules). In a further preferred embodiment, the HLA-A molecule may be selected from HLA-A*02 and HLA-A*02:01.

The template peptide is, for example relative to the desired peptide, typically a low-affinity peptide with low off-rate at the reduced temperature (and high off-rate at the increased temperature), and that in a temperature-dependent manner can be exchanged for exogenous peptides of interest (desired peptide).

The skilled person understands, within the context of the current invention how to prepare a MHC molecule, such as a HLA-A molecule, having bound thereto in the peptide-binding groove of said MHC molecule a template peptide that dissociates from said MHC molecule at an increased temperature, for example using such methods as described in the Example, for example using such methods as described by Toebes et al. (Current Protocols in Immunology 18.16.1-18.16.20, 2009), both for monomers and multimers.

In the next step, the temperature of the MHC molecule, preferably a HLA-A molecule, with the template peptide bound is increased to an increased temperature. It was found that preferably temperature may be increased either gradually and step-wise, for example using a 0.05-5 degrees Celsius step gradient, preferably an about 1° C. step gradient with 10 seconds-60 seconds, preferably about 30 second temperature stabilization for each step.

In another embodiment it was found that the MHC molecule such as a MHC class I molecule, preferably a HLA-A molecule, with the template peptide may also be brought to the increased temperature directly, without applying a temperature gradient, i.e. in one step, for example by placing the MHC molecule such as a HLA-A molecule with the template peptide under conditions of the increased temperature.

It was surprisingly found that this step can successfully be performed both using monomers and using multimers (e.g. using complexes comprising at least two MHC molecules, preferably HLA-A molecules).

Either way, the temperature of the MHC molecule(s), preferably the HLA-A molecule(s) with the template peptide is brought from the reduced temperature to the increased temperature, thereby causing the dissociation of the template peptide from the MHC molecule.

A next part of the method of the invention comprises contacting the MHC molecule, preferably the HLA-A molecule(s), at the increased temperature with a desired peptide for binding to the peptide-binding groove of said MHC molecule, under conditions allowing the desired peptide to bind to the peptide-binding groove of said MHC molecule.

It will be understood by the skilled person that the desired peptide is a peptide that is expected associate with the MHC molecule, preferably a HLA-A molecule, at the increased temperature whereas, at the same time the template peptide dissociates from said MHC molecule, effectively replacing the template peptide with the desired peptide.

The desired peptide may be any peptide as long as it may bind in the peptide-binding groove of the MHC molecule/associate with the MHC molecule, preferably a HLA-A molecule.

Indeed, for example, the template peptide and/or the desired peptide comprises from about 7 to about 12 amino acids, preferably 8, 9 or 10 amino acids, when the MHC molecule is a MHC class I molecule, preferably a HLA-A molecule, or the template peptide and/or the desired peptide comprises from about 15 to 30 amino acids when the MHC molecule is a MHC class II molecule.

The desired peptide may, for example, be a known, expected or unknown antigenic peptide, including neo-antigenic antigen/epitope.

The current invention is not in particular limited with respect to whether the MHC molecule (such as MHC class I, preferably a HLA-A molecule), with the template peptide is contacted with the desired peptide once the increased temperature is applied to the MHC molecule, or that the desired peptide is already provided to the MHC molecule loaded with the template peptide before the increased temperature is applied, for example by contacting the MHC molecule with the template peptide with the desired peptide already at the reduced temperature or during the application of a temperature gradient, for example as described herein elsewhere.

Preferably the desired peptide is first contacted with the MHC molecule (such as a MHC class I molecule, preferably a HLA-A molecule), loaded with the template peptide under conditions under which the template peptide does not dissociate from the MHC molecule, followed by increasing the temperature to the increased temperature. In such embodiment, steps b) and c) are performed at the same time, i.e. simultaneously//.

It will be understood by the skilled person that the desired peptide will have a higher affinity for the MHC molecule, preferably a HLA-A molecule, than the template peptide used, in particular at the increased temperature. At the increased temperature, the template peptide has a high off-rate whereas the desired peptide has a low(er) off-rate.

The period of contacting the MHC molecule, preferably a HLA-A molecule, with the desired peptide is not in particular limited, and, as will be understood by the skilled person, may depend on the MHC molecule (e.g. in the case of a HLA-A molecule), the template peptide and the desired peptide used. The skilled person understands how to optimize both the temperature and the period of contact. However, in some embodiments, and with increasing preference, step b) or step b) and c) is performed for a period of between 1 minute and 6 hours, for a period of between 2 minutes and 3 hours, for a period of between 5 minutes and 180 minutes, for example for about 2 minutes, 5 minutes, 10 minutes, 20 minutes, 50 minutes, 60 minutes, 90 minutes, 180 minutes, 270 minutes, or more.

Although, in view of general principle of the method as claimed herein, the invention is not in particular limited with respect to the “reduced temperature” and the “increased temperature”, according to some embodiments, the reduced temperature is a temperature of 10 degrees Celsius or less and/or the increased temperature is a temperature of 15 degrees Celsius or more, preferably wherein the reduced temperature is 4 degrees Celsius or less and/or wherein the increased temperature is between, and including, 20 degrees Celsius and 40 degrees Celsius.

For example, in embodiments of the current invention, the reduced temperature is a temperature, that is, or is below, with increasing preference, 11 degrees Celsius, 9 degrees Celsius, 8 degrees Celsius, 6 degrees Celsius, or 4 degrees Celsius. Preferably the reduced temperature is above −10 degrees Celsius, −5 degrees Celsius, −1 degree Celsius, or 0 degree Celsius. For example, the MHC molecule with the template peptide may be provide in step a) on ice.

In some preferred embodiments, the reduced temperature is, with increasing preference, between, and including, 0 degrees Celsius and 10 degrees Celsius, 0 degrees Celsius and 8 degrees Celsius, 0 degrees Celsius and 6 degrees Celsius, or 0 degrees Celsius and 4 degrees Celsius.

For example, in embodiments of the current invention, the increased temperature is, or is above, 17 degrees Celsius, 20 degrees Celsius, 22 degrees Celsius, 25 degrees Celsius, 28 degrees Celsius, 30 degrees Celsius, 32 degrees Celsius, 35 degrees Celsius, 37 degrees Celsius, 40 degrees Celsius, 45 degrees Celsius, 50 degrees Celsius, 55 degrees Celsius, or 60 degrees Celsius. For example, the MHC molecule with the template peptide may be subjected to room temperature (e.g. between 18 degrees Celsius and 22 degrees Celsius).

Preferably, the increased temperature is no more than, with increasing preference, 65 degrees Celsius, 60 degrees Celsius, 55 degrees Celsius, 50 degrees Celsius, 45 degrees Celsius or 40 degrees Celsius.

In some preferred embodiments the increased temperature is, with increasing preference, between and including, 15 degrees Celsius and 60 degrees Celsius, 17 degrees Celsius and 50 degrees Celsius, 20 degrees Celsius and 45 degrees Celsius, or 22 degrees Celsius and 40 degrees Celsius.

It was found that exchange of the template peptide with the desired peptide can advantageously be performed when the difference between said reduced temperature and said increased temperature is at least 5 degrees Celsius, 10 degrees Celsius, 15 degrees Celsius, 20 degrees Celsius, 25 degrees Celsius, or 30 degrees Celsius, for example between 5 degrees Celsius and 50 degrees Celsius, between 8 degrees Celsius and 40 degrees or between 10 degrees Celsius and 30 degrees Celsius

The skilled person will understand that the increased temperature is a temperature at which the template peptide dissociates from the MHC molecule (such as MHC class I, preferably a HLA-A molecule), with such rate that the desired peptide can associate with the MHC molecule. It needs no explanation that the increased temperature is not a temperature that is too high to allow the desired peptide to effectively associate with the MHC molecule (preferably a HLA-A molecule), causing the MHC molecule to become unstable. In fact, in some embodiments, it is preferred that the increased temperature, i.e. the temperature at which the exchange of the template peptide with the desired peptide is to be performed is as low as possible (i.e. the template peptide should still dissociate, and the desired peptide should still associate), in particular in case desired peptides with relative low affinity are used.

For example, the MHC molecule, preferably a HLA-A molecule, with the template peptide bound thereto denatures when brought to the increased in the absence of the desired peptide, preferably at least 95%, 96%, 97%, 98%, 99%,100% of the MHC molecule with the template peptide bound thereto denatures in the absence of the desired peptide.

It was found that with the method of the current invention, the desired peptide may replace, with increasing preference, at least 90%, 91%, 92%, 93%, 94%, 95%, 96%, 97%, 98%, 99%, or 100% of the template peptide at the increased temperature.

It was found that the template peptide dissociates, with increasing preference, for 95%, 96%, 97%, 98%, 99%, or 100% from the MHC molecule, preferably a H LA-A molecule, at the increased temperature.

At the same time it was found that, with the method of the current invention, loss of properly folded or functional MHC molecules (such as MHC class I molecules, particularly HLA-A molecules), during the exchange can be reduced or prevented. In other words, high yields of MHC molecule (particularly HLA-A molecules), including multimers, loaded with the desired peptide can be obtained. For example, loss of less than about 30%, 25%, 20%, 15%, 10%, 8%, 7%, 6%, 5%, 4%, 3% or 2% of the initial amount or number of (properly folded) MHC molecule (e.g. HLA-A molecules loaded with the template peptide) may be achieved. In other words, yields of about 70%, 75%, 85%, 90%, 92%, 93%, 94%, 95%, 96%, 97% or 98% relative to the initial amount or number of (properly folded) MHC molecule (e.g. HLA-A molecules loaded with the template peptide) may be achieved.

In embodiments of the current invention, the desired peptide is provided in step c) in excess of the MHC molecule, preferably a HLA-A molecule, with the template peptide bound thereto, preferably wherein the excess is at least about 5-fold, 10-fold 20-fold, 30-fold, 50-fold, 100-fold, 200-fold molar excess. It was found that by providing such molar excess high yields of properly exchanged MHC molecules, preferably HLA-A molecules, (including multimers) are obtained.

At the same time, it was found that that the input peptides used (template peptides as well as the desired peptide to be introduced) are preferably substantially pure before (e.g. comprises, with increasing preference, less than 1% (w/w), 0.9% (w/w), 0.8% (w/w), 0.7% (w/w), 0.6% (w/w), 0.5% (w/w), 0.4% (w/w), 0.3% (w/w) of another peptide, in particular another peptide with an affinity for the MHC molecule (preferably a HLA-A molecule) higher than the intended input peptide), for example are pure (0.1-0.0 (w/w)). Indeed, since a large excess of peptide compared to MHC molecule (e.g. MHC heavy chain) is used, a small impurity may result in incorrect refolding of a large portion of the MHC, e.g. MHC I. In some experiments, it was found that such impurity with a peptide with affinity for MHC (preferably HLA-A) higher than the intended input peptide, may result in a stable batch of peptide-MHC complexes that could not be exchanged anymore.

As explained herein, the method of the invention can be applied using MHC monomers (such as MHC class I monomers, preferably HLA-A monomers), but also, and with preference, using MHC multimers (such as MHC class I multimers, preferably HLA-A multimers). In the latter case, the MHC multimers loaded with a template peptide are provided in step a) and steps b) and c) may be performed directly using such multimers, and importantly without the need of an additional step of multimerization of MHC monomers. Indeed in case in step a) MHC monomers (preferably HLA-A monomers) are provided and steps b) and c) are performed using such monomers, and in case multimers are desired (preferably HLA-A multimers), after step c) the monomers needs to be subjected to multimerization, for example using methods known in the art.

Therefore, in a highly preferred embodiment of the method of the current inventions, the MHC molecule (such as MHC I molecule, preferably HLA-A molecule) of step a), having bound thereto in the peptide-binding groove of said MHC molecule a template peptide that dissociates from said MHC molecule at an increased temperature, is in the form of a multimer (preferably HLA-A multimer). In the multimer, preferably at least one, two, three or all of the MHCs, have bound thereto in the peptide-binding groove of the MHC a template peptide that dissociates at an increased temperature. In some embodiments, the MHC, preferably a HLA-A molecule, may be in the form of a complex comprising at least two MHC molecules (preferably two HLA-A molecules).

In some embodiments, the MHC molecule (such as MHC class I, preferably a HLA-A molecule), is part of a complex comprising the MHC molecule and at least one other molecule, preferably at least one other protein, preferably at least one other MHC molecule. The MHC molecule (preferably HLA-A molecule), the template peptide and/or the desired peptide may be provided, for example by covalent linkage, with addition groups of chemical moieties, including labels such as fluorescent labels or chromophores and the like.

In some embodiments, the multimer is a MHC-dimer, MHC-trimer, MHC-tetramer, MHC-pentamer, MHC-hexamer of MHC-decamer, wherein the MHC molecule is preferably a HLA-A molecule. An example are the multimers provided by Immudex (www.immudex.com//about-products/dextramer-descrip.aspx)

Although the invention may be applied utilizing any type of MHC molecules, it is contemplated that the MHC molecule is, with increasing preference, a mammalian MHC molecule, a human MHC molecule or human leukocyte antigen (HLA), a MHC class I molecule, human HLA-A, HLA-A*02, or HLA-A*02:01 (HLA-A*02 is a human leukocyte antigen serotype within the HLA-A serotype group).

In certain embodiment, when the MHC molecule is from mice, the MHC molecule is preferably H-2K^{b}.

Although the template peptide provided in the MHC molecule, preferably a HLA-A molecule, of step a) is not in particular limited, except for its characteristic of having a low off-rate from the MHC molecule at the reduced temperature, while effectively dissociating from the MHC molecule at the increased temperature, it was found that in some preferred embodiments the template peptide is obtained by substitution of at least one, two or more anchor residues, preferably of one or two anchor residues in a known ligand or antigenic peptide/epitope for said MHC molecule. Antigenic peptides bind the MHC molecule through interaction between such anchor amino acids on the peptide and relevant domains of the MHC molecule. Anchor residues are known to the skilled person and are found in for example both MHC Class I (.e.g. HLA-A) and Class II binding peptides. Indeed MHC I (e.g. HLA-A) and class II molecules fold into a highly similar conformations featuring the peptide-binding groove to present T-cell epitopes. Peptide-binding grooves of MHC I molecules are composed of two α-helices and eight β-strands formed by one heavy chain, while MHC II uses two domains from different chains to construct the peptide-binding groove. The peptides bind to MHC molecules through primary and secondary anchor residues protruding into the pockets in the peptide-binding grooves (See, Major Histocompatibility Complex: Interaction with Peptides by Liu et al. DOI: 10.1002/9780470015902.a0000922.pub2). Anchor residues and motifs are known for most MHC molecules (Rammensee H et al (1999) SYFPEITHI: database for MHC ligands and peptide motifs. Immunogenetics 50(3-4):213-219).

By replacing one, two or more of the anchor residues in a known ligand, peptides, suitable as template peptides for use in the method of the current invention may be obtained. Preferably, the template peptide for use in the method according to the invention is obtained by substitution of anchor residue(s) in a known ligand with known affinity for smaller amino acids. The skilled person understand what in the context of the current invention smaller amino acids are. In general, the bigger an anchor amino acid the more interaction it has with the MHC. Within the context of the current invention, it was found that decreasing the size of the amino acid reduces the amount of interactions with the MHC (preferably a HLA-A molecule) and may provide for a peptide suitable as template peptide. In some embodiments the substitution is within the same functional amino acid group (e.g. hydrophobic, or charged).

For example, for ILKEPVHGV, as used in the example herein—anchor residues L at position 2 and V at position 9 are both hydrophobic. When considering the amino acids sizes (for example, http://people.mbi.ucla.edu/sawaya/m230d/Modelbuilding/aadensity.png), Alanine (A) is the smallest hydrophobic amino acid, so it is good substitute for both Leucine (L) and Valine (V) therefore the resulting successful template peptides are IAKEPVHGV or IAKEPVHGA. Alternatively, one could substitute Leucine for Valine resulting in peptide IVKEPVHGV or IVKEPVHGA to have peptide of higher predicted affinity than IAKEPVHGV or IAKEPVHGA, but which may be suitable as template peptide.

In one embodiment, and for in particular for HLA-A*02:01, the amino acid on positions 2 and/or 9 (for example in case of a known ligand peptide with length 9) or positions 2 and/or 10 (for example in case of a known ligand peptide with length 10) (see the Immune Epitope Database and Analysis resource for HLA-A*02:01 (http://www.iedb.org/MHCalleleld/143)) are replaced by an amino acid that is smaller in size. The skilled person will understand, that based on public available date, and in similar fashion, the anchor residues in other MHC molecules, such as other HLA-A*02 or HLA-A molecules, may likewise be replaced as a potential way to provide for a template peptide suitable for use in the methods according to the invention. As is exemplified in the examples herein, the desired peptide to be exchanged with the template peptide does not have to be related (based on e.g. amino acid sequence similarity of the peptides) to the template peptide and may be of unrelated structure.

In a preferred embodiment the template peptide (as used in the Example disclosed herein) is a polypeptide comprising

a. the polypeptide sequence as set forth in SEQ ID NO: 1 (IAKEPVHGV), SEQ ID NO: 2 (IAKEPVHGA) or SEQ ID NO:3 (FAPGNAPAL); or

b. the polypeptide sequence as set forth in SEQ ID NO:1 or SEQ ID NO:2 or SEQ ID NO:3 having 1, 2, 3, or 4 amino acid substitutions, deletions or insertions.

As shown in the Examples, the HLA-A*02:01-IAKEPVHGV complex, HLA-A*02:01-IAKEPVHGA complex or H-2K^{b}-FAPGNAPAL complex are MHC molecules loaded with a template peptide that can suitably be used in the method of the invention.

It will be understood by the skilled person that the polypeptide sequences as set forth in SEQ ID NO:1 (IAKEPVHGV), SEQ ID NO:2 (IAKEPVHGA) or SEQ ID NO:3 (FAPGNAPAL) may comprise further substitutions in 1, 2, 3 or 4 amino acids without departing from the spirit of the invention.

A major advantage of the method of the current invention is that the MHC molecules (such as MHC class I molecules, preferably HLA-A molecules), in particular multimers (preferably HLA-A multimers), provided with the template peptide may be stored at low temperatures (as discussed herein elsewhere) or may be prepared in bulk in advance of performing the method of the current invention. In addition, the method of the current invention only requires changing the temperature from the reduced temperature to the increased temperature, in the presence of the desired peptide, as discussed herein in detail. These elements of the method according to the invention makes the method in particular suitable for performing the assay in parallel for a number of desired peptides, for example using multi-well systems, and wherein a MHC molecule (preferably a HLA-A molecule) having a template peptide is contacted with a different peptide in each of the used wells, of with a different concentration of the same peptide in various wells, of with a combination of different peptides, of with a combination of a peptide and a further compound, for example in order to study the modulation effect of such compound on exchange of the template peptide with the desired peptide. This is in particular advantageous when the MHC molecule with the template peptide is a multimer.

Also provided is for a method according to the invention wherein the MHC molecule (such as MHC class I, preferably a HLA-A molecule), provided in step a), preferably a multimer, is produced and loaded with the template peptide at the reduced temperature. The skilled person is well aware of methods to provide for such MHC molecule (preferably HLA-A molecule), including multimers. For example, the MHC molecule (preferably HLA-A molecule) having bound thereto in the peptide-binding groove of said MHC molecule a template peptide is provided by refolding of a MHC molecule at a temperature of 10 degrees or less in the presence of the template peptide.

In some embodiments, the method is performed in a system that is free of any cells. In some embodiments the method is an in vitro method.

In some embodiments, the method further comprises detecting binding of said desired peptide to said MHC molecule (such as a MHC class I molecule, preferably a HLA-A molecule), preferably wherein said binding is detected by detecting a label that is associated with said desired peptide, preferably wherein said desired peptide comprises said label.

Such method is for example useful for diagnostic purposes. Binding can be detected in various ways, for instance via T cell receptor or antibody specific for said peptide presented in the context of said MHC molecule (such as a MHC class I molecule, preferably a HLA-A molecule). Binding is preferably detected by detecting a label that is associated with the desired peptide. This can be done by tagging the peptide with a specific binding molecule, for example with biotin that can subsequently be visualized via for instance, labelled streptavidin.

In a preferred embodiment said peptide comprises said label. In this way any peptide bound to said MHC-molecule (such as MHC class I molecule, preferably HLA-A molecule) can be detected directly. Detection of binding is preferably done for screening purposes, preferably in a high throughput setting. Preferred screening purposes are screening for compounds that affect the binding of said peptide to said MHC molecule. For instance, test peptides or small molecules can compete with binding of said peptide to said MHC molecule. Competition can be detected by detecting decreased binding of said peptide.

Likewise and in a similar fashion, template peptide binding or dissociation may be detected, using detecting a label that is associated with said template peptide, preferably wherein said template peptide comprises said label.

As explained herein, also provided is for the method of the invention, for determining binding of said desired peptide in the presence of a test or reference compound.

According to another aspect of the invention, there is provided for the MHC molecule (such as MHC class I, preferably HLA-A molecule) obtainable with the method as disclosed herein. Also provided is for a composition comprising such MHC molecule obtainable with the method of the invention and T cells, preferably CD8⁺ T cells.

Also provided is for a MHC molecule (such as MHC class I, preferably HLA-A molecule), at a temperature of 10 degrees of less and having bound thereto in the peptide-binding groove of said MHC molecule a template peptide that dissociates from said MHC molecule when the temperature is 15 degrees Celsius, preferably when the temperature is between 15 degrees Celsius and 40 degrees Celsius.

Also provided is for a MHC molecule (such as MHC class I, preferably HLA-A molecule), preferably at a temperature of 10 degrees of less, having bound thereto in the peptide-binding groove of said MHC molecule a template peptide wherein the template peptide is a polypeptide comprising
a. the polypeptide sequence as set forth in SEQ ID NO:1 (IAKEPVHGV), SEQ ID NO:2 (IAKEPVHGA) or SEQ ID NO:3 (FAPGNAPAL); or
b. the polypeptide sequence as set forth in SEQ ID NO:1 or SEQ ID NO:2 or SEQ ID NO:3 having 1, 2, 3, or 4 amino acid substitutions, deletions or insertions.

As shown in the Examples, the HLA-A*02:01-IAKEPVHGV complex, HLA-A*02:01-IAKEPVHGA complex or H-2K^{b}-FAPGNAPAL complex are MHC molecules loaded with a template peptide that can suitable used in the method of the invention.

It will be understood by the skilled person that the polypeptide sequences as set forth in SEQ ID NO:1 (IAKEPVHGV), SEQ ID NO:2 (IAKEPVHGA) or SEQ ID NO:3 (FAPGNAPAL) may comprise further substitutions in 1, 2, 3 or 4 amino acids without departing from the spirit of the invention.

Also provided is for a composition comprising such MHC molecule (such as MHC class I, preferably HLA-A molecule), preferably at a temperature of 10 degrees of less, having bound thereto in the peptide-binding groove of said MHC molecule a template peptide. In some embodiments, the composition may further comprise a further peptide, preferably wherein said further peptide is an antigenic peptide capable of binding in peptide-binding groove of the MHC molecule, for example the desired peptide as used herein. In some embodiments, the composition further comprises NaCl, preferably 100-600 mM NaCl, more preferably 250-350 mM NaCl and/or glycerol, preferably 1-50% (vol/vol) glycerol, preferably 5-15% (vol/vol) glycerol. In particular this is advantageous when the composition is a composition is stored at low temperature (e.g. below 0 degrees Celsius). Therefore, also provided is for a composition stored at a temperature of, with increasing preferences, less than 10 degrees Celsius, less than 0 degrees Celsius, less than −20 degrees Celsius wherein the composition comprises an MHC molecule (preferably HLA-A molecule) having bound thereto in the peptide-binding groove of said MHC molecule a template peptide that dissociates from said MHC molecule at a temperature of 15 degrees Celsius or more, preferably when the temperature is between 15 degrees Celsius and 40 degrees Celsius, and preferably further comprises NaCl, preferably 100-600 mM NaCl, more preferably 250-350 mM NaCl and/or glycerol, preferably 1-50% (vol/vol) glycerol, preferably 5-15% (vol/vol) glycerol; preferably wherein the MHC molecule is a multimer.

Also provided is for a template peptide that binds with a MHC molecule (such as MHC class I, preferably a HLA-A molecule) at the reduced temperature but not at the increased temperature. In some embodiments, the template peptide is a polypeptide comprising
a. the polypeptide sequence as set forth in SEQ ID NO:1, SEQ ID NO:2 or SEQ ID NO:3; or
b. the polypeptide sequence as set forth in SEQ ID NO:1 or SEQ ID NO:2 or SEQ ID NO:3 having 1, 2, 3, or 4 amino acid substitutions, deletions or insertions

Also provided is for the use of the template peptide of above for producing a MHC molecule (such as MHC class I, preferably a HLA-A molecule), and/or for use in peptide exchange of a MHC molecule (preferably a HLA-A molecule).

Also provided is for the use of a MHC molecule (such as MHC class I, preferably a HLA-A molecule) having bound thereto in the peptide-binding groove of said MHC molecule a template peptide that dissociates from said MHC molecule at an increased temperature for producing a MHC molecule, and/or for use in peptide exchange of a MHC molecule.

Also provided is for the use of composition comprising a MHC molecule (such as a MHC class I molecule, preferably a HLA-A molecule) as obtained with the method of the invention, for detecting T cells recognizing the desired peptide.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### SEQUENCES USED HEREIN

(SEQ ID NO: 1)
IAKEPVHGV
(SEQ ID NO: 2)
IAKEPVHGA
(SEQ ID NO: 3)
FAPGNAPAL
(SEQ ID NO: 4)
SIINFEKL
(SEQ ID NO: 5)
FAPGNWPAL
(SEQ ID NO: 6)
FAPGNYPAA
(SEQ ID NO: 7)
FAPGNAPAL
(SEQ ID NO: 8)
FAPGNYPAL
(SEQ ID NO: 9)
ILKEPVHGV
(SEQ ID NO: 10)
ILKEPVHGA
(SEQ ID NO: 11)
WLIGFDFDV
(SEQ ID NO: 12)
SGYNFSLGAAV
(SEQ ID NO: 13)
SSPPMFRV
(SEQ ID NO: 14)
RALEYKNL
(SEQ ID NO: 15)
NLVPMVATV
(SEQ ID NO: 16)
VLEETSVML
(SEQ ID NO: 17)
CLGGLLTMV
(SEQ ID NO: 18)
GLCTLVAML
(SEQ ID NO: 19)
YVLDHLIVV
(SEQ ID NO: 20)
LLDQLIEEV

### EXAMPLES

Example 1

### Temperature-Induced Peptide Exchange on MHC Multimers for Antigen-Specific T Cell Detection

### GENERAL INTRODUCTION

### Introduction

We set-out to develop a faster, more convenient technology for peptide exchange on multimers. We surprisingly found that such technology may be provided by the design of low-affinity peptides with low off-rate at reduced temperature, e.g. at 4° C., and that in a temperature-dependent manner can be exchanged for exogenous peptides of interest. We provide proof-of-concept for H-2K^{b} and HLA-A*02:01 multimers, representatives of dominant mouse and human MHC alleles, respectively. We have performed peptide exchange on pre-folded MHC multimers that could be used ad hoc to measure T cell kinetics against various viral reactivations in a transplant recipient. Temperature-exchangeable MHC I multimers will provide convenient tools for epitope discovery and immune monitoring.

Our technology can be used for the production of MHC multimers for immunodiagnostics; immune monitoring, isolation of epitope-specific T cells, for anti-viral or cancer therapy, or in general epitope identification to study behavior and evolution of the immune system.

### MATERIALS AND METHODS

### Peptide Synthesis and Purification

Peptides were synthesized in our lab by standard solid-phase peptide synthesis in N-methyl-2-pyrrolidone using Syro I and Syro II synthesizers. Amino acids and resins were used as purchased from Nova Biochem. Peptides were purified by reversed phase HPLC using a Waters HPLC system equipped with a preparative Waters X-bridge C18 column. The mobile phase consisted of water acetonitrile mixtures containing 0.1% TFA. Peptide purity and composition were confirmed by LC-MS using a Waters Micromass LCT Premier G2-XS QT of mass spectrometer equipped with a 2795 separation module (Alliance HT) and 2996 photodiode array detector (Waters Chromatography B.V.). LC-MS samples were run over a Kinetex C18 column (Phenomenex, United States, CA) in a water/acetonitrile gradient. Analysis was performed using MassLynx 4.1 software (Waters Chromatography). Peptides were purified twice if necessary.

### Protein Expression and Purification

MHC class I (MHC I) complexes were expressed and refolded according to previously published protocols²⁵. Refolded complexes of H-2K^{b} were purified twice using anion exchange (0 to 1M NaCl in 20 mM Tris-HCl pH 8; Resource Q column) and size exclusion chromatography (150 mM NaCl, 20 mM Tris-HCl pH 8; Superdex 75 16/600 column) on an ÄKTA (GE Healthcare Life Sciences) or NGC system (Bio-Rad). We discovered that recovery was considerably lower when purifying using anion exchange and size exclusion chromatography, as compared to using size exclusion only, possibly caused by strong interaction between peptide and ion-exchange resin. Therefore, to maximize purification yields, refolded complexes of HLA-A*02:01 were purified using only size exclusion chromatography (300 mM NaCl, 20 mM Tris-HCl pH8). Purified properly folded complexes were concentrated using Amicon Ultra-15 30 kDa MWCO centrifugal filter units (Merck Millipore), directly biotinylated using BirA ligase where needed, purified again using size exclusion chromatography and stored in 300 mM NaCl, 20 mM Tris-HCl (pH 8) with 12.5% glycerol at −80° C. until further use.

### Protein Unfolding

Thermal unfolding of different H-2K^{b}- and HLA-A*02:01-peptide complexes was determined using an Optim 1000 (Avacta Analytical Ltd) machine. MHC I-peptide complexes were measured in 150 mM NaCl, 20 mM Tris-HCl (pH 7.5) buffer or phosphate-buffered saline (PBS) at a protein concentration of 0.2 mg/ml. Samples were heated using a 1° C. step gradient with 30 s temperature stabilization for each step. Unfolding was followed by measuring tryptophan fluorescence emission at a range from 300 to 400 nm following excitation at 266 nm. Barycentric fluorescence was determined according to the equation:

BCMλ=(ΣI(λ)×λ)/(ΣI(λ))

where BCMλ is the Barycentric mean fluorescence in nm, I(λ) is the fluorescence intensity at a given wavelength, and λ is the wavelength in nm.

The melting temperature (Tₘ) was calculated using Barycentric fluorescence as a function of temperature according to the equation:

$${T}_{m} = max \frac{dBMC}{d t} ( T )$$

where max is the local maximum and

$$\frac{dBCM}{dt} ( T )$$

is the first derivative of Barycentric fluorescence as a function of temperature in

$$[ \frac{nm}{° C .} ] .$$

Analysis was performed with Optim Analysis Software v 2.0 (Avacta Analytical Ltd).

### Multimerization of MHC I Monomers

MHC I monomers were complexed with allophycocyanin (APC)- or phycoerythrin (PE)-labeled streptavidin to form multimers for T cell analysis. Typically, temperature-labile peptide-MHC complexes were multimerized on ice by stepwise addition of fluorochrome-labeled streptavidin with one minute intervals. Full biotinylation was verified by HPLC. Aliquots of multimers were snap frozen in 150 mM NaCl, 20 mM Tris-HCl pH 7.5 containing 15% glycerol. For T cell staining the desired peptide in PBS was added to the multimer solution while thawing to obtain a final concentration of 0.5 μM MHC and 50 μM peptide.

### Analysis of Temperature-Mediated Peptide Exchange

To initiate peptide exchange 0.5 μM MHC I-peptide complex was incubated with 50 μM exchange peptide in 110 μl PBS under defined exchange conditions. After incubation exchange solutions were centrifuged at 14,000×g for 1 min at RT and subsequently the supernatant was analyzed by gel filtration on a Shimadzu Prominence HPLC system equipped with a 300×7.8 mm BioSep SEC-s3000 column (Phenomenex) using PBS as mobile phase. Data were processed and analyzed using Shimadzu LabSolutions software (version 5.85).

### Relative Exchange Efficiency Determined by Mass Spectrometry

In order to quantify peptide exchange on H-2K^{b}, 0.5 μM H-2K^{b} monomers (H-2K^{b}-FAPGNAPAL were incubated with 50 μM peptide SIINFEKL (SEQ ID NO: 4), FAPGNWPAL (SEQ ID NO: 5), FAPGNYPAA (SEQ ID NO: 6), or FAPGNAPAL (SEQ ID NO: 7) in PBS for 45 min at room temperature. In order to quantify peptide exchange on HLA-A*02:01, 0.5 μM HLA-A*02:01 monomers were incubated with 50 μM of peptide in PBS for 3 hours at 32° C.

Before analysis, exchanged monomers were spun at 14,000×g for 1 min at room temperature to remove aggregates and subsequently purified using a Microcon-30kDa Centrifugal Filter Unit with Ultracel-30 membrane (Merck Millipore, pre-incubated with tryptic BSA digest to prevent stickiness of the peptides to the membrane) to remove unbound excess peptide. After washing twice with PBS and twice with ammonium bicarbonate at room temperature, MHC-bound peptides were eluted by the addition of 200 μl 10% acetic acid followed by mixing at 600 rpm for 1 min at room temperature. Eluted peptides were separated using a Microcon-30 kDa Centrifugal Filter Unit with Ultracel-30 membranes. Eluates were lyophilized and subjected to mass spectrometry analysis.

For MS analysis, peptides were dissolved in 95/3/0.1 v/v/v water/acetonitrile/formic acid and subsequently analyzed by on-line nanoHPLC MS/MS using an 1100 HPLC system (Agilent Technologies), as described previously²⁶. Peptides were trapped at 10 μl/min on a 15-mm column (100-μm ID; ReproSil-Pur C18-AQ, 3 μm, Dr. Maisch GmbH) and eluted to a 200 mm column (50-μm ID; ReproSil-Pur C18-AQ, 3 μm) at 150 nl/min. All columns were packed in house. The column was developed with a 30-min gradient from 0 to 50% acetonitrile in 0.1% formic acid. The end of the nanoLC column was drawn to a tip (5-μm ID), from which the eluent was sprayed into a 7-tesla LTQ-FT Ultra mass spectrometer (Thermo Electron).

The mass spectrometer was operated in data-dependent mode, automatically switching between MS and MS/MS acquisition. Full scan MS spectra were acquired in the FT-ICR with a resolution of 25,000 at a target value of 3,000,000. The two most intense ions were then isolated for accurate mass measurements by a selected ion-monitoring scan in FT-ICR with a resolution of 50,000 at a target accumulation value of 50,000. Selected ions were fragmented in the linear ion trap using collision-induced dissociation at a target value of 10,000. To quantify the amount of eluted peptide standard curves were created with the respective synthetic peptides.

### Mice

Wild-type (WT) C57BL/6 mice (Charles River) were maintained at the Central Animal Facility of the Leiden University Medical Center (LUMC) under specific pathogen-free conditions. Mice were infected intraperitoneally with 5×10⁴ PFU murine cytomegalovirus (MCMV)-Smith (American Type Culture Collection (ATCC) VR-194; Manassas, Va.), derived from salivary gland stocks from MCMV-infected BALB/c mice, or with 2×10⁵ PFU lymphocytic choriomeningitis virus (LCMV) Armstrong propagated on baby hamster kidney (BHK) cells. Virus titers were determined by plaque assays as published²⁷. All animal experiments were performed with approval of the Animal Experiments Committee of the LUMC and according to the Dutch Experiments on Animals Act that serves the implementation of ‘Guidelines on the protection of experimental animals’ by the Council of Europe and the guide to animal experimentation set by the LUMC.

### Collection of Primary Human Material

Peripheral blood samples were obtained from a HLA-A*02:01-positive multiple myeloma patient after T cell-depleted allogeneic stem cell transplantation (allo-SCT), after approval by the LUMC and written informed consent according to the Declaration of Helsinki. To monitor viral reactivation Epstein-Barr virus (EBV) and HCMV DNA loads on fresh whole blood were assessed by quantitative polymerase chain reaction (qPCR). Peripheral blood mononuclear cells (PBMCs) were collected using Ficoll Isopaque separation (LUMC Pharmacy, Leiden, The Netherlands) and cryopreserved in the vapor phase of liquid nitrogen. Virus-specific CD8⁺ T cell reconstitution was determined on thawed PBMCs by flow cytometry.

### Antibodies and Reagents

Ficoll Isopaque was obtained from the LUMC Pharmacy (Leiden, The Netherlands). Fluorochrome-conjugated antibodies were purchased from several suppliers. V500 anti-mouse CD3, FITC anti-mouse CD8, FITC anti-human CD4, Pacific Blue anti-human CD8, APC anti-human CD14 were purchased from Becton Dickinson (BD) Biosciences. BV605 anti-mouse CD8 was purchased from BioLegend. Fluorochrome-conjugated streptavidin and 7-AAD were purchased from Invitrogen. DAPI was purchased from Sigma. Conventional HLA-A*02:01 PE-labeled tetramers were produced as described previously for all indicated T cell specificities¹¹. Human interleukin-2 (IL-2) was purchased from Chiron (Amsterdam, The Netherlands). Human serum albumin (HSA) was purchased from Sanquin Reagents (Amsterdam, The Netherlands).

### Flow Cytometry Analysis of Murine CD8⁺ T Cells

H-2K^{b}-FAPGNAPAL multimers were exchanged for selected peptides for 5 min at RT and subsequently used for staining of the H-2K^{b}-restricted OVA₂₅₇₋₂₆₄-specific TCR transgenic line (OT-I), described previously²⁸. Generally, 200,000 cells were stained first with APC- or PE-labeled temperature-exchanged or conventional multimers for 10 min at RT and then with surface marker antibodies (anti-CD8-FITC) at 4° C. for 20 min. Cells were washed twice with and then resuspended in FACS buffer (0.5% BSA and 0.02% sodium azide in PBS). DAPI was added at a final concentration of 0.1 μg/ml. Samples were measured using a BD FACSAria Fusion and data were analyzed with BD FACSDiva software (version 8.0.2).

Virus-specific T cells were analyzed in blood samples of LCMV-infected mice after erythrocyte lysis or splenocytes obtained from MCMV-infected, 8-10 weeks old mice (infected at 6-8 weeks). Erythrocytes were lysed using a hypotonic ammonium chloride buffer (150 mM NH₄Cl, 10 mM KHCO₃; pH 7.2+/−0.2). Cells were simultaneously stained with appropriate temperature-exchanged multimers and surface markers (7-AAD, anti-CD3-V500, anti-CD8-BV605) for 30 min at 4° C. Multimers were titrated to establish optimal T cell staining. Generally, a dilution of 1:20-1:40 was sufficient to stain 10,000-100,000 T cells in 50 μl FACS buffer. Cells were washed twice with and resuspended in FACS buffer. Sample data were acquired using a BD Fortessa flow cytometer and analyzed using BD FACSDiva software (version 8.0.2).

### Flow Cytometry Analysis of Human CD8⁺ T Cells

Multimers of HLA-A*02:01-IAKEPVHGV (SEQ ID NO: 1) were exchanged for selected peptides at 32° C. for 3 h and used to stain corresponding CD8⁺ T cells. UV-exchanged multimers were produced and exchanged following published protocols^{17,18}.

Clones or cell lines of the indicated viral T cell specificities (cultured in Iscove's Modified Dulbecco's Medium (IMDM) supplemented with 10% human serum and 100 IU/ml IL-2) were mixed with PBMCs of a HLA-A*02:01-negative donor to be able to discriminate multimer-positive from multimer-negative cells. Following incubation with PE-labeled temperature-exchanged, conventional multimers or UV-exchanged multimers for 10 min at 4° C., cells were stained with surface marker antibodies (anti-CD8-Pacific Blue, anti-CD14-APC) for 20 min on ice. Multimers were titrated to establish optimal T cell staining without background. Cells were washed twice with and resuspended in FACS buffer (0.5% HSA in PBS). Samples were acquired using a BD FACSCanto II flow cytometer and analysis was performed with BD FACSDiva software (version 8.0.2). The absolute numbers of multimer positive CD8⁺ T cells were calculated based on the percentage of multimer positive cells within the CD8⁺ T cell population and the concentration of CD8⁺ T cells in whole blood.

### RESULTS

### Identification of MHC I-Peptide Pairs Suitable for Temperature Exchange

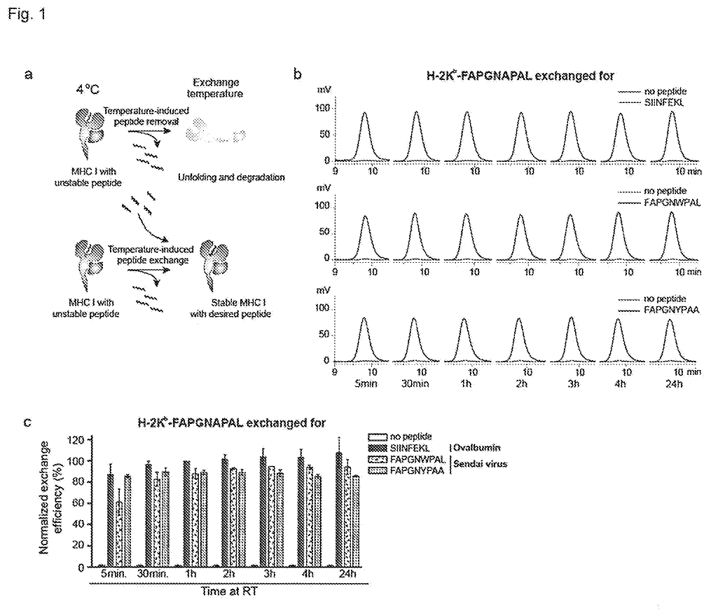

When designing peptides suitable for MHC, for example MHC I, temperature exchange the most important criterion identified is that the MHC I complex loaded with a conditional ligand (template peptide) should be stable at low temperatures, but unstable at higher temperatures, for example, it should efficiently refold at 4° C., but upon an increase in temperature allow peptide dissociation and binding of incoming peptide cargo (FIG. 1a). The main determinant for MHC I-peptide stability is the peptide off-rate from MHC I²³. We identified peptides known to bind to the respective MHC I molecules with low off-rates and substituted their anchor residues to increase off-rates. It was found that the input peptides (template peptides as well as the desired peptide to be introduced) are preferably pure before adding them to refolding reactions. Since a large excess of peptide compared to MHC heavy chain is used, even an almost undetectable impurity can be preferentially selected by the refolding MHC I to yield complexes with unexpected stabilities (data not shown).

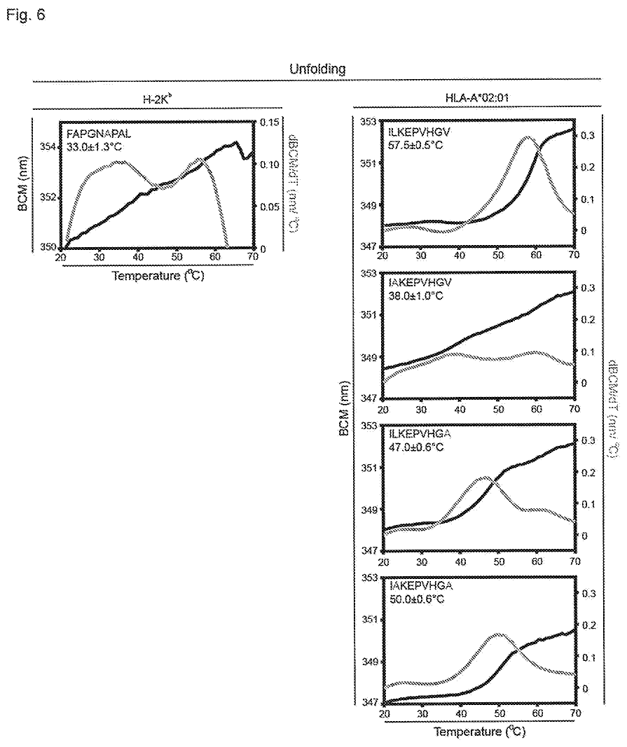

We have previously produced murine H-2K^{b} complexes with low-affinity peptides derived from the Sendai virus epitope FAPGNYPAL (SEQ ID NO: 8) (NP₃₂₄₋₃₃₂) and analyzed their stability and kinetics of peptide binding²³. We found that from the seven peptides tested, only FAPGNAPAL fulfilled the criteria required for peptide exchange. The melting temperature of the H-2K^{b} complex with FAPGNAPAL, defined as midpoint of thermal denaturation, is ˜33° C. (FIG. 6). In line with this, FAPGNAPAL swiftly dissociated from and did not rebind to H-2K^{b} at either of the two elevated temperatures tested (26° C. and 32° C.)²³. This indicates that the H-2K^{b}-FAPGNAPAL complex is sufficiently stable to refold at 4° C., but unstable at elevated temperatures and could therefore be a suitable complex for temperature-induced peptide exchange.

In order to translate the exchange technology to human applications, we set out to identify a suitable peptide for HLA-A*02:01, the most frequent human MHC I allele in the Caucasian population. We designed peptides based on the HIV-1 epitope ILKEPVHGV (SEQ ID NO: 9) (RT₄₇₆₋₄₈₄) with one (IAKEPVHGV or ILKEPVHGA (SEQ ID NO: 10) or both anchors (IAKEPVHGA) modified. HLA-A*02:01 complexes with modified peptides were produced and thermal stability experiments carried out, where tryptophan fluorescence was monitored over a temperature range to assess HLA-A*02:01-peptide complex unfolding. Surprisingly, out of the four complexes tested HLA-A*02:01-IAKEPVHGV showed the lowest melting temperature (˜38° C.) (FIG. 6). We found that the melting temperature is a first indication that HLA-A*02:01-IAKEPVHGV could be suitable for temperature-based peptide exchange.

### Temperature-Labile MHC I-Peptide Monomers Efficiently Exchange for a Range of Peptides

We next evaluated the exchange efficiency of H-2K^{b} in complex with FAPGNAPAL over a temperature range using analytical size exclusion HPLC. We found that the complex is unstable at room temperature (20° C.), resulting in denaturation and precipitation. This is illustrated by the absence of an MHC I peak when analyzed by HPLC (FIG. 1b). When incubated in the presence of a high affinity peptide (SIINFEKL, OVA₂₅₇₋₂₆₄) a clear peak was observed, demonstrating that H-2K^{b} could be “rescued” from unfolding (FIG. 1b, upper panel). Exchange of FAPGNAPAL (K_{D}>4 μM²³) for SIINFEKL (K_{D}=1.4 nM²⁹) was almost complete within 30 min. the efficiency increased only by 15% after 24 h (FIG. 1b, upper panel and 1c).

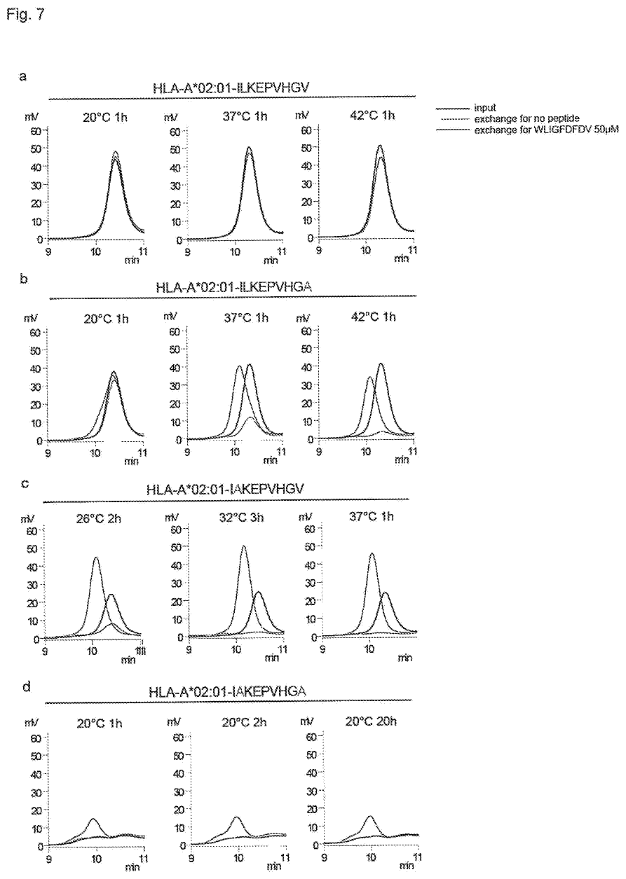

Similarly, HLA-A*02:01 in complex with either of four peptides based on ILKEPVHGV were tested for exchange with a high affinity binding peptide (vaccinia virus (VACV) B19R-A2WLIGFDFDV, K_{D}=0.06 nM³⁰) (SEQ ID NO: 11) at different temperatures and time points. HLA-A*02:01 in complex with ILKEPVHGV or ILKEPVHGA remained stable at room temperature and even at elevated temperatures intact HLA-A*02:01 could still be detected (37 or 42° C., FIG. 7a-b). Considering also their high melting temperatures (˜57 and 47° C., respectively, FIG. 6), and dissociation constants (ILKEPVHGV−K_{D}=2.5 nM³¹; ILKEPVHGA−K_{D}=1.1 μM predicted with NetMHC^{32, 33}), ILKEPVHGV and ILKEPVHGA fail as input peptides in the exchange reaction.

We continued the search for optimal peptides binding to HLA-A*02:01 allowing efficient temperature-induced exchange. Complexes of HLA-A*02:01 with IAKEPVHGV (K_{D}=7.3 μM predicted with NetMHC^{32, 33}) or IAKEPVHGA (K_{D}=19.1 μM predicted with NetMHC^{32, 33}) peptides were considerably less stable, even at room temperature (FIG. 7c-d). As a result of higher stability, the refolding efficiency of HLA-A*02:01-IAKEPVHGV was substantially higher than that of HLA-A*02:01-IAKEPVHGA (Table 1), as was maximum rescue (FIG. 7c-d).

TABLE 1
Refolding efficiencies of ILKEPVHGV-derived HLA-
A*02:01-peptide complexes. Refolding efficiencies
represented as a percentage of purified properly
folded HLA-A*02:01-peptide complex related to
input free heavy chain (from inclusion bodies).

| Peptide | Refolding efficiency (%) |
|---|---|
| ILKEPVHGV | 2.6 |
| IAKEPVHGV | 5.7 |
| ILKEPVHGA | 3.7 |
| IAKEPVHGA | 0.9 |

HLA-A*02:01-IAKEPVHGV was efficiently exchanged at two temperatures: at 37° C. for 1 h or at 32° C. for 3 h (FIG. 7c). We selected HLA-A*02:01-IAKEPVHGV as the best candidate complex for general peptide exchange applications, despite its higher temperature required for optimal exchange.

In conclusion, we have identified two MHC I-peptide pairs allowing efficient temperature-induced exchange reactions. Our selection criteria for defining optimal exchange complexes should be extendable to other MHC alleles.

As a broad technology, MHC I multimers should exchange their peptides for many different peptides, including those with a relatively low affinity, such as many cancer antigen-derived peptides³⁴. To test the broad applicability of this technology, we exchanged FAPGNAPAL for either FAPGNWPAL (K_{D}=33 nM at 26° C. and K_{D}=33 nM at 32° C.²³) or FAPGNYPAA (K_{D}=18 nM at 26° C. and K_{D}=144 nM at 32° C.²³). For both suboptimal peptides, the exchange efficiency reached 80-90% of the level observed for SIINFEKL (FIG. 1b-c). Mass spectrometry analysis showed that exchange complexes contained 94.2% of FAPGNWPAL and 84.4% of FAPGNYPAA, respectively. After exchange no template peptide FAPGNAPAL was detected, which demonstrates that all MHC I-peptide complexes contained the exchanged peptide (Table 2).

TABLE 2
Relative quantification of exchange efficiency by
mass spectrometry. Peptide exchange on MHC I was
performed with 0.5 μM monomers (H-2K^{b} or HLA-
A*02:01), incubated with 50 μM of peptide as
explained in Online Methods. Monomers were also
incubated in the absence of peptide to determine
the stability of the complexes under these
conditions. To quantify the amount of eluted
peptide standard curves were created with the
respective synthetic peptides. H-2K^{b}-SIINFEKL was
measured as positive control.

| H-2K^{b} monomer | Peptide | Efficiency of exchange |
|---|---|---|
| folded with | exchanged for | CYO |
| FAPGNAPAL | SIINFEKL | 105.5 ± 4.7 |
| | FAPGNWPAL | 94.2 ± 10.8 |
| | FAPGNYPAA | 84.4 ± 6.2 |
| | FAPGNAPAL | 4.2 ± 0.1 |
| | — | 0.1 ± 0.1 |
| SIINFEKL | — | 107.4 ± 12.6 |

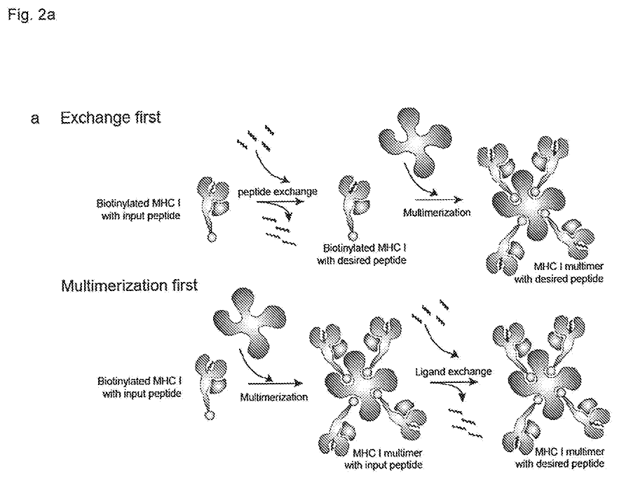

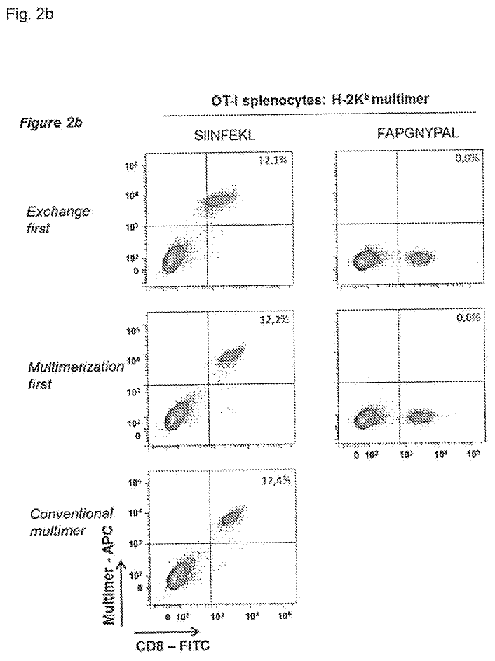

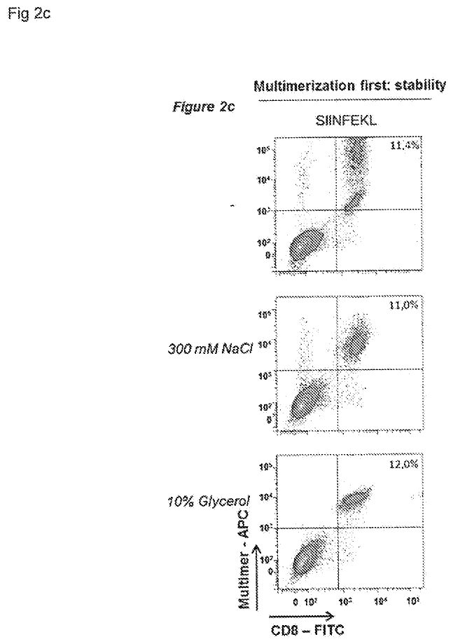

Detection of antigen-specific CD8⁺ T cells using ready-to-use temperature-exchanged MHC I multimers The technology of peptide exchange would be even more attractive if it could be applied directly on MHC I multimers, a severe limitation of current parallel exchange technology. In current exchange technologies monomers are first exchanged and then multimerized (FIG. 2a, upper panel), but the method described here can also be applied directly to multimers (FIG. 2a, lower panel). To test the functionality of exchanged multimers, we generated multimers either after or before peptide exchange and used these to stain SIINFEKL-specific OT-I T cells (FIG. 2b). Multimers prepared by temperature exchange performed indistinguishably from conventional multimers (FIG. 2b). No positive staining was observed when multimers were not exchanged (data not shown) or exchanged for an irrelevant peptide (FAPGNYPAL, FIG. 2b).

When assessing multimer stability upon freezing, we found that multimers alone suffered from freeze-thaw cycles, but addition of about 300 mM NaCl and/or about 10% glycerol before freezing ensured stability during freeze-thaw cycles (FIG. 2c). We conclude that temperature-mediated peptide exchange can therefore be used to produce MHC multimers ready for loading with diverse sets of peptides directly from temperature-exchangeable multimer stocks. This represents a significant advantage by taking away any time-consuming preparation preceding multimer staining experiments.

The immune responses to LCMV and MCMV infections in C57BL/6 mice have been extensively characterized and we used these infections as a model to illustrate the quality of our temperature-exchanged multimers in the detection of antigen-specific CD8⁺ T cells³⁵⁻³⁸.

We measured the CD8⁺ T cell responses to the following immunodominant epitopes: LCMV epitope NP238-K^{b}/SGYNFSLGAAV (SEQ ID NO: 12) and MCMV epitopes M38-K^{b}/SSPPMFRV (SEQ ID NO: 13) and IE3-K^{b}/RALEYKNL (SEQ ID NO: 14) (Table 3).

TABLE 3
Peptides used in this study and descriptions of their modifications. Some of
the peptides used are derivatives of FAPGNYPAL or ILKEPVHGV modified at anchor
positions (indicated in bold). HAdV - human adenovirus, LCMV - Lymphocytic
Choriomengitis Virus, CMV - cytomegalovirus, HIV - human immunodeficiency virus, EBV -
Epstein-Barr virus, VACV - vaccinia virus, m - mouse, h - human, affinity to the respective
MHC is either from published evidence, or predicted with NetMHC (indicated with *)

| MHC I | | | | |
|---|---|---|---|---|
| allele | Source | Epitope | Sequence | K_{D} (nM) |
| H-2K^{b} | Ovalbumin | OVA₂₅₇₋₂₆₄ | SIINFEKL | 1.4²⁹ |
| | Sendai | NP₃₂₄₋₃₃₂ | FAPGNYPAL | 108²³ |
| | virus | | FAPGNWPAL | 31²³ |
| | | | FAPGNYPAA | 144²³ |
| | | | FAPGNAPAL | >4000²³ |
| | LCMV | NP₂₃₈₋₂₄₈ | SGYNFSLGAAV | 0.38⁴³ |
| | MCMV | M38₃₁₆₋₃₂₃ | SSPPMFRV | 392*^{32,33} |
| | | IE3₄₁₆₋₄₂₃ | RALEYKNL | 23*^{32,33} |
| HLA- | VACV | B19R₂₉₄₋₃₀₂ | WLIGFDFDV | 0.06³⁰ |
| A*02:01 | HIV-1 | RT₄₇₆₋₄₈₄ | ILKEPVHGV | 2.5³¹ |
| | | | IAKEPVHGV | 7,288*^{32,33} |
| | | | ILKEPVHGA | 1,095*^{32,33} |
| | | | IAKEPVHGA | 19,111*^{32,33} |
| | HCMV | pp65₄₉₅₋₅₀₃ | NLVPMVATV | 26*^{32,33} |
| | | IE-1₃₁₆₋₃₂₄ | VLEETSVML | 297*^{32,33} |
| | EBV | BMLF-1₂₈₀₋ | GLCTLVAML | 139*^{32,33} |
| | | LMP2₄₂₆₋₄₃₄ | CLGGLLTMV | 76*^{32,33} |
| | | BRLF-1₁₀₉₋ | YVLDHLIVV | 4.1*^{32,33} |
| | HAdV | E1A₁₉₋₂₇ | LLDQLIEEV | 16*^{32,33} |

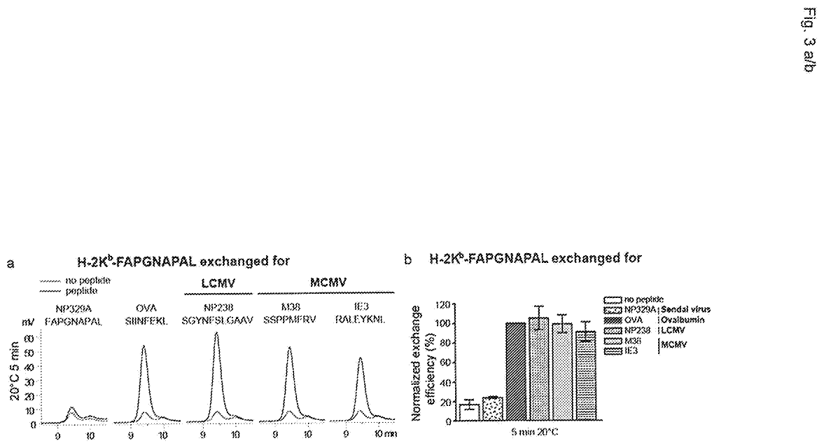

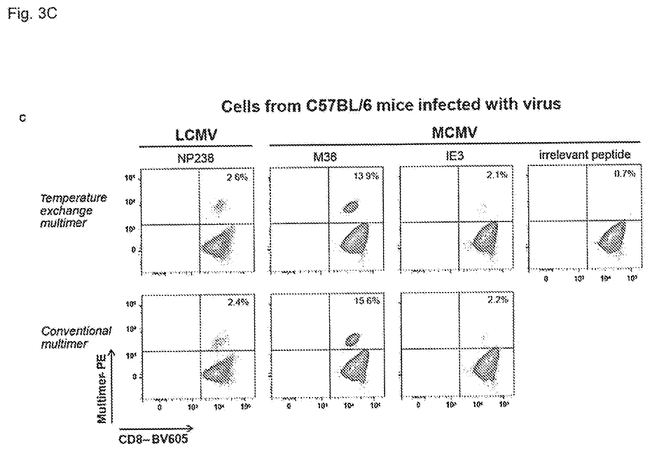

We first measured exchange on H-2K^{b} monomers by HPLC. As for SIINFEKL, all three peptides exchanged with high efficiency within 5 min at room temperature and produced stable H-2K^{b} complexes, which was not observed for exchange reactions without peptide or with an excess of FAPGNAPAL (FIG. 3a, quantified in 3b). Subsequently, we performed temperature-mediated exchange for these three viral epitopes on H-2K^{b} multimers and used these multimers to stain blood samples from LCMV-infected mice or splenocytes from MCMV-infected mice.

Within 5 min after taking the multimers with temperature-sensitive peptides from the freezer, the multimers were ready and stained antigen-specific CD8⁺ T cells as efficiently as conventional multimers (FIG. 3c), demonstrating the applicability of temperature exchange technology.

Likewise, HLA-A*02:01-IAKEPVHGV monomers could be readily exchanged for selected viral epitopes (HCMV pp65-A2/NLVPMVATV (SEQ ID NO: 15), HCMV IE-1-A2/VLEETSVML (SEQ ID NO: 16), EBV LMP2-A2/CLGGLLTMV (SEQ ID NO: 17), EBV BMLF-1-A2/GLCTLVAML (SEQ ID NO: 18), EBV BRLF1-A2/YVLDHLIVV (SEQ ID NO: 19) and human adenovirus (HAdV) E1A-A2/LLDQLIEEV (SEQ ID NO: 20), details in Table 3, when incubated at 32° C. for 3 h or 37° C. for 45 min.

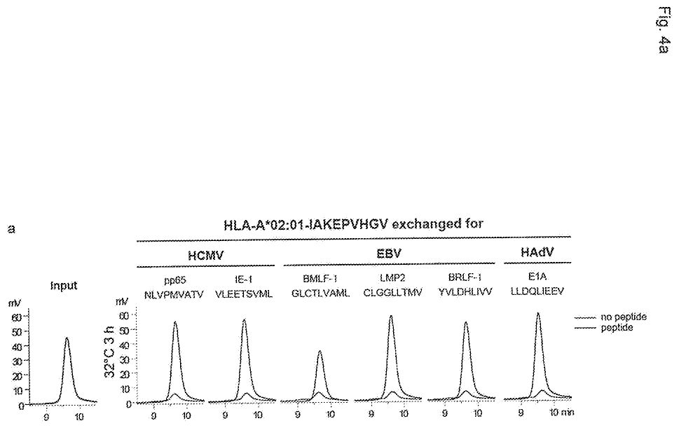

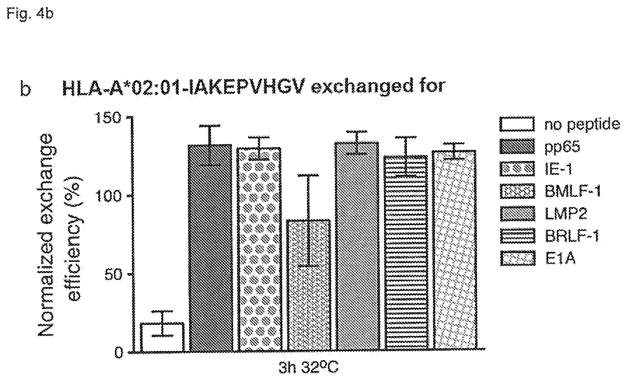

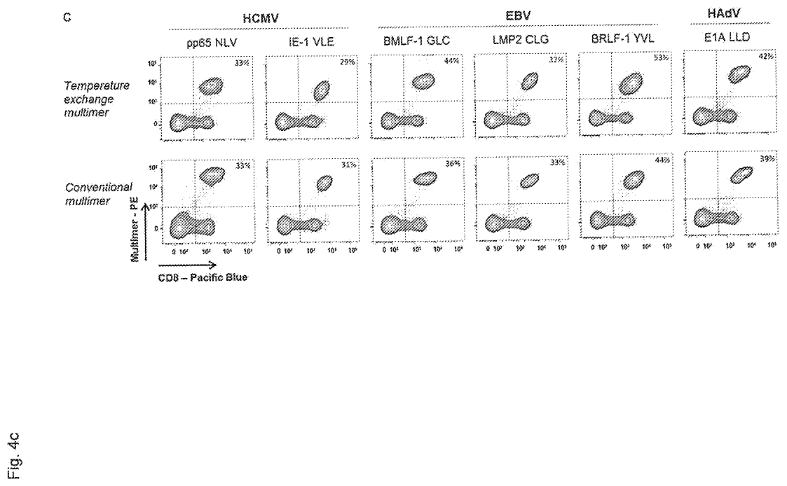

HPLC analysis showed that following incubation at 32° C. without peptide no MHC peak was detected, indicating degradation and precipitation of MHC monomers (FIG. 4a). However, after incubation with peptide the peak area of MHC I monomers was at least as high as that of non-incubated complexes for all peptides (FIG. 4a, quantified in 4b). Incubation at 37° C. for 45 min likewise resulted in efficient rescue.

To be able to exchange for peptides across a wide spectrum of affinities we selected 3 h at 32° C. as optimal exchange condition for HLA-A*02:01.

Multimers exchanged for these epitopes were ready within 3 h and used directly to stain CD8⁺ T cell clones with corresponding specificities. Detected percentages of multimer-positive CD8⁺ T cells corresponded to those detected using either conventional or UV-exchanged multimers, confirming their proper function (FIG. 4c). No staining was observed when incubated with multimers exchanged for irrelevant peptides.

### Exchanged MHC I-Peptide Multimers are Effective Reagents for Immunomonitoring

To demonstrate the value of our reagents also in clinical practice, we compared our temperature-exchanged multimers with conventional multimers in an immune monitoring setting. Because after T cell-depleted allogeneic stem cell transplantation (allo-SCT) patients are heavily immunocompromised, T cell reconstitution is of major importance to prevent morbidity and mortality caused by opportunistic herpesvirus infections like HCMV and EBV^{2, 3}. Therefore, patients are intensively monitored until a donor-derived immune system has developed.

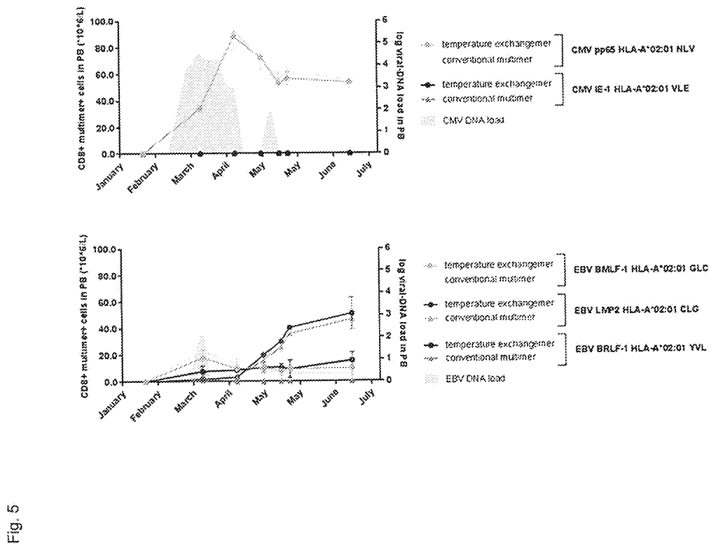

We exchanged PE-labeled HLA-A*02:01-IAKEPVHGV multimers for a selection of HCMV and EBV epitopes in parallel and used these to stain peripheral blood mononuclear cells (PBMCs) obtained after allo-SCT at weekly intervals to monitor T cell frequencies. The kinetics of CD8⁺ T cells specific for HCMV pp65-A2/NLV are in concordance with the HCMV reactivation illustrated by the expansion of HCMV viral DNA (FIG. 5, upper panel). Although a positive EBV DNA load was measured only once, T cells specific for EBV LMP2-A2/CLG and to a lesser extent those specific for EBV BMLF-1-A2/GLC expanded over time (FIG. 5, lower panel). No significant responses were detected against HCMV IE-1-A2/VLE (FIG. 5, upper panel) or EBV BRLF1-A2/YVL (FIG. 5, lower panel). Indeed this is patient-specific. Since there were no T cells specific for these epitopes detected using conventional multimers this confirms that the multimers provided with the method as disclosed herein are specific. Frequencies of specific T cells were comparable between conventional and temperature-exchanged multimers. This further emphasizes the efficiency and flexibility of our technology to rapidly produce many different MHC I multimers ad hoc for the detection of antigen-specific T cells, even at the low frequencies typically found in primary immune monitoring samples.

### DISCUSSION

Here we describe a surprising but reliable approach that allows the parallel generation of large sets of different MHC multimers. Our approach can be applied in all laboratories, since it only requires a −80° C. freezer for storage of exchangeable multimer stocks and a thermoblock, water bath or PCR machine for incubation at the optimal temperature for exchange. This system is faster and less laborious than the generation of multimers from single MHC I-peptide combinations, both those made by producing each complex by refolding and purification, as well as those generated by chemically-triggered or UV-mediated peptide exchange¹⁴⁻¹⁷.

The approach allows fast and near quantitative peptide exchange on multimers, whereas parallel multimer generation using UV-mediated exchange is variable due to uneven evaporation across and between sample plates and cannot be performed on ready-made MHC I multimers due to fluorophore bleaching.

We have established a method where ready-made temperature-sensitive MHC I multimers can be stored at −80° C. and while thawing can ad hoc be incubated with peptides of choice to allow peptide exchange within 5-180 minutes, depending on the MHC I allele. This is the most robust technique for multimer production developed to date, that will facilitate immunomonitoring and discovery of new (neo) antigens. We anticipate that rapid, robust, and inexpensive detection of MHC-antigen-specific T cells will have a strong impact on the immunomonitoring of responses to infection, but also responses to vaccines against cancer and infectious diseases, as well as on cancer immunotherapy^{22, 39-41}.

We have shown for two MHC I alleles, one murine and one human, that temperature-exchanged multimers could as efficiently as conventional- or UV-exchanged multimers stain specific CD8⁺ T cells, including those present at low frequencies. The design of peptides suitable for temperature exchange on HLA-A*02:01 proved more challenging than H-2K^{b}, partly because of the intrinsically higher stability of human MHC class I complexes compared to murine MHC I. We have demonstrated for both H-2K^{b}-FAPGNAPAL and HLA-A*02:01-IAKEPVHGV that the temperature-labile input peptide may be exchanged for both high- and low-affinity peptides, making it possible to test for a broad array of T cell specificities. MHC multimers temperature-exchanged for low-affinity peptides are highly specific, as no difference in background stain as compared to conventional or UV-exchanged multimers was observed. Their use in monitoring viral reactivation in an allo-SCT recipient illustrates the flexibility and straightforwardness of temperature-exchangeable MHC I multimers.

We designed peptides to form stable complexes with MHC I at low temperatures that can be released at elevated temperatures. The selection of optimal peptides allowing low temperature exchange and full replacement by exogenous peptides, is not obvious. A number of options include peptides with suboptimal length, smaller anchor residues and altered N- or C-termini²⁴. Even then, many peptide sequences have to be tested to identify the optimal MHC I-peptide combination, as we describe here for the most frequently used mouse and human MHC I alleles. Yet, expanding this principle to the many other MHC I alleles could provide a procedure where viral or tumor antigens are sequenced, the fragments that may bind are predicted and synthesized within a day, and loaded on the ready-to-use MHC I multimers (as stored in the −80° C. freezer). Within two days a patient's T cell responses could then be monitored, as the production of the MHC I multimers is no longer the time limiting factor.

In conclusion, we present a fast and easy method for the generation of MHC I multimers loaded with epitopes at wish. This method will render MHC multimer technology accessible to any research or clinical chemistry laboratory and this may become the method of choice.

Having now fully described this invention, it will be appreciated by those skilled in the art that the same can be performed within a wide range of equivalent parameters, concentrations, and conditions without departing from the spirit and scope of the invention and without undue experimentation.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### LITERATURE CITED

1. Grinde, B. Herpesviruses: latency and reactivation—viral strategies and host response. J Oral Microbiol 5(2013).
2. Broers, A. E., et al. Increased transplant-related morbidity and mortality in CMV-seropositive patients despite highly effective prevention of CMV disease after allogeneic T-cell-depleted stem cell transplantation. Blood 95, 2240-2245 (2000).
3. Green, M. L., et al. Cytomegalovirus viral load and mortality after haemopoietic stem cell transplantation in the era of pre-emptive therapy: a retrospective cohort study. Lancet Haematol 3, e119-127 (2016).
4. Hodi, F. S., et al. Improved survival with ipilimumab in patients with metastatic melanoma. N Engl J Med 363, 711-723 (2010).
5. Page, D. B., Yuan, J. & Wolchok, J. D. Targeting cytotoxic T-lymphocyte antigen 4 in immunotherapies for melanoma and other cancers. Immunotherapy 2, 367-379 (2010).
6. Tumeh, P. C., et al. PD-1 blockade induces responses by inhibiting adaptive immune resistance. Nature 515, 568-571 (2014).
7. Topalian, S. L., et al. Safety, activity, and immune correlates of anti-PD-1 antibody in cancer. N Engl J Med 366, 2443-2454 (2012).
8. Robert, L., et al. Distinct immunological mechanisms of CTLA-4 and PD-1 blockade revealed by analyzing TCR usage in blood lymphocytes. Oncoimmunology 3, e29244 (2014).
9. van Rooij, N., et al. Tumor exome analysis reveals neoantigen-specific T-cell reactivity in an ipilimumab-responsive melanoma. J Clin Oncol 31, e439-442 (2013).
10. Fourcade, J., et al. PD-1 is a regulator of NY-ESO-1-specific CD8+ T cell expansion in melanoma patients. J Immunol 182, 5240-5249 (2009).
11. Altman, J. D., et al. Phenotypic analysis of antigen-specific T lymphocytes. Science 274, 94-96 (1996).
12. Ljunggren, H. G., et al. Empty MHC class I molecules come out in the cold. Nature 346, 476-480 (1990).
13. Saini, S. K., et al. Dipeptides catalyze rapid peptide exchange on MHC class I molecules. Proc Natl Acad Sci USA 112, 202-207 (2015).
14. Amore, A., et al. Development of a hypersensitive periodate-cleavable amino acid that is methionine- and disulfide-compatible and its application in MHC exchange reagents for T cell characterisation. Chembiochem 14, 123-131 (2013).
15. Rodenko, B., et al. Class I major histocompatibility complexes loaded by a periodate trigger. J Am Chem Soc 131, 12305-12313 (2009).
16. Choo, J. A., et al. Bioorthogonal cleavage and exchange of major histocompatibility complex ligands by employing azobenzene-containing peptides. Angew Chem Int Ed Engl 53, 13390-13394 (2014).
17. Rodenko, B., et al. Generation of peptide-MHC class I complexes through UV-mediated ligand exchange. Nat Protoc 1, 1120-1132 (2006).
18. Toebes, M., et al. Design and use of conditional MHC class I ligands. Nat Med 12, 246-251 (2006).
19. Bakker, A. H., et al. Conditional MHC class I ligands and peptide exchange technology for the human MHC gene products HLA-A1, -A3, -A11, and -B7. Proc Natl Acad Sci USA 105, 3825-3830 (2008).
20. Hadrup, S. R., et al. Parallel detection of antigen-specific T-cell responses by multidimensional encoding of MHC multimers. Nat Methods 6, 520-526 (2009).
21. Andersen, R. S., et al. Dissection of T-cell antigen specificity in human melanoma. Cancer Res 72, 1642-1650 (2012).
22. Bentzen, A. K., et al. Large-scale detection of antigen-specific T cells using peptide-MHC-I multimers labeled with DNA barcodes. Nat Biotechnol 34, 1037-1045 (2016).
23. Garstka, M. A., et al. The first step of peptide selection in antigen presentation by MHC class I molecules. Proc Natl Acad Sci USA 112, 1505-1510 (2015).
24. De Silva, A. D., et al. Thermolabile H-2Kb molecules expressed by transporter associated with antigen processing-deficient RMA-S cells are occupied by low-affinity peptides. J Immunol 163, 4413-4420 (1999).
25. Toebes, M., Rodenko, B., Ovaa, H. & Schumacher, T. N. Generation of peptide MHC class I monomers and multimers through ligand exchange. Curr Protoc Immunol Chapter 18, Unit 18 16 (2009).
26. Meiring, H. D., van der Heeft, E., ten Hove, G. J. & de Jong, A. P. J. M. Nanoscale LC-MS(n): technical design and applications to peptide and protein analysis. J Sep Sci 25, 557-568 (2002).
27. Welten, S. P. M., et al. The viral context instructs the redundancy of costimulatory pathways in driving CD8(+) T cell expansion. Elife 4(2015).
28. Hogquist, K. A., et al. T cell receptor antagonist peptides induce positive selection. Cell 76, 17-27 (1994).
29. Vitiello, A., et al. Immunodominance analysis of CTL responses to influenza PR8 virus reveals two new dominant and subdominant Kb-restricted epitopes. J Immunol 157, 5555-5562 (1996).
30. Ishizuka, J., et al. Quantitating T cell cross-reactivity for unrelated peptide antigens. J Immunol 183, 4337-4345 (2009).
31. Madden, D. R., Garboczi, D. N. & Wiley, D. C. The antigenic identity of peptide-MHC complexes: a comparison of the conformations of five viral peptides presented by HLA-A2. Cell 75, 693-708 (1993).
32. Andreatta, M. & Nielsen, M. Gapped sequence alignment using artificial neural networks: application to the MHC class I system. Bioinformatics 32, 511-517 (2016).
33. Nielsen, M., et al. Reliable prediction of T-cell epitopes using neural networks with novel sequence representations. Protein Sci 12, 1007-1017 (2003).
34. Duan, F., et al. Genomic and bioinformatic profiling of mutational neoepitopes reveals new rules to predict anticancer immunogenicity. J Exp Med 211, 2231-2248 (2014).
35. Rodriguez, F., Slifka, M. K., Harkins, S. & Whitton, J. L. Two overlapping subdominant epitopes identified by DNA immunization induce protective CD8(+) T-cell populations with differing cytolytic activities. J Virol 75, 7399-7409 (2001).
36. Matloubian, M., Concepcion, R. J. & Ahmed, R. CD4+ T cells are required to sustain CD8+ cytotoxic T-cell responses during chronic viral infection. J Virol 68, 8056-8063 (1994).
37. Wherry, E. J., Blattman, J. N., Murali-Krishna, K., van der Most, R. & Ahmed, R. Viral persistence alters CD8 T-cell immunodominance and tissue distribution and results in distinct stages of functional impairment. J Virol 77, 4911-4927 (2003).
38. van der Most, R. G., et al. Identification of Db- and Kb-restricted subdominant cytotoxic T-cell responses in lymphocytic choriomeningitis virus-infected mice. Virology 240, 158-167 (1998).
39. La Rosa, C., et al. MVA vaccine encoding CMV antigens safely induces durable expansion of CMV-specific T cells in healthy adults. Blood 129, 114-125 (2017).
40. Grassmann, A. A., et al. Discovery of Novel Leptospirosis Vaccine Candidates Using Reverse and Structural Vaccinology. Front Immunol 8, 463 (2017).
41. El Bissati, K., et al. Adjuvanted multi-epitope vaccines protect HLA-A*11:01 transgenic mice against Toxoplasma gondii. JCI Insight 1, e85955 (2016).
42. Sahin, U., et al. Personalized RNA mutanome vaccines mobilize poly-specific therapeutic immunity against cancer. Nature 547, 222-226 (2017).
43. Kotturi, M. F., et al. The CD8(+) T-cell response to lymphocytic choriomeningitis virus involves the L antigen: Uncovering new tricks for an old virus. Journal of Virology 81, 4928-4940 (2007).
Glithero, A. et al. Crystal Structures of Two H-2Db/Glycopeptide Complexes Suggest a Molecular Basis for CTL Cross-Reactivity. Immunity 10, 63-74 (1999)

What is claimed is:

1. A method for producing a MHC molecule, the method comprising
a. Providing at a reduced temperature an MHC molecule having bound thereto in the peptide-binding groove of said MHC molecule a template peptide that dissociates from said MHC molecule at an increased temperature, wherein said MHC molecule is preferably a human leukocyte antigen—A (HLA-A) molecule;
b. Changing the temperature to an increased temperature, therewith dissociating the template peptide from said MHC molecule; and
c. Contacting the MHC molecule at said increased temperature with a desired peptide for binding to the peptide-binding groove of said MHC molecule, under conditions allowing the desired peptide to bind to the peptide-binding groove of said MHC molecule.

2. The method of claim 1, wherein the reduced temperature is a temperature of 10 degrees Celsius or less and/or the increased temperature is a temperature of 15 degrees Celsius or more, preferably wherein the reduced temperature is 4 degrees Celsius or less and/or wherein the increased temperature is between, and including, 20 degrees Celsius and 40 degrees Celsius.

3. The method of claim 1, wherein b) and c) are performed simultaneously.

4. The method of claim 1, wherein the desired peptide is provided in excess of the MHC molecule with the template peptide bound thereto, preferably wherein the excess is at least about 5-fold, 10-fold 20-fold, 30-fold, 50-fold, 100-fold, 200-fold molar excess.

5. The method of claim 1, wherein the MHC molecule in step a) is provided as a monomer, as a complex comprising at least two MHC molecules, or as a multimer.

6. The method of claim 1, wherein the MHC molecule is part of a complex comprising the MHC molecule and at least one other molecule, preferably at least one other protein, preferably at least one other MHC molecule.

7. The method of claim 1, wherein the MHC molecule is a human HLA-A molecule, and wherein said HLA-A molecule is preferably selected from HLA-A*02 and HLA-A*02:01.

8. The method of claim 1, wherein the template peptide is obtained by substitution of at least one, two or more anchor residues, preferably of one or two anchor residues.

9. The method of claim 1, wherein the template peptide is a polypeptide comprising
a. the polypeptide sequence as set forth in SEQ ID NO:1 (IAKEPVHGV), SEQ ID NO:2 (IAKEPVHGA) or SEQ ID NO:3 (FAPGNAPAL); or
b. the polypeptide sequence as set forth in SEQ ID NO:1 or SEQ ID NO:2 or SEQ ID NO:3 having 1, 2, 3, or 4 amino acid substitutions, deletions or insertions.

10. The method of claim 1, wherein the method is performed in parallel for different desired peptides for binding to the peptide-binding groove of said MHC molecule.

11. The method of claim 1 wherein the MHC molecule provided in step a) is produced and loaded with the template peptide at the reduced temperature.

12. The method of claim 1, wherein the MHC molecule having bound thereto in the peptide-binding groove of said MHC molecule a template peptide is provided by refolding of a MHC molecule at a temperature of 10 degrees or less in the presence of the template peptide.

13. The method of claim 1 wherein the method is cell-free.

14. The method of claim 1 further comprising detecting binding of said desired peptide to said MHC-molecule, preferably wherein said binding is detected by detecting a label that is associated with said desired peptide, preferably wherein said desired peptide comprises said label.

15. The method of claim 1, for determining binding of said desired peptide in the presence of a test or reference compound.

16-22. (canceled)

23. A template peptide that binds with a MHC molecule at the reduced temperature but not at the increased temperature, wherein the MHC molecule is preferably a human HLA-A molecule, preferably a human HLA-A molecule selected from HLA-A*02 and HLA-A*02:01.

24. A template peptide of claim 23 wherein the template peptide is a polypeptide comprising
a. the polypeptide sequence as set forth in SEQ ID NO:1 (IAKEPVHGV), SEQ ID NO:2 (IAKEPVHGA) or SEQ ID NO:3 (FAPGNAPAL); or
b. the polypeptide sequence as set forth in SEQ ID NO:1 or SEQ ID NO:2 or SEQ ID NO:3 having 1, 2, 3, or 4 amino acid substitutions, deletions or insertions

25-27. (canceled)

28. A composition stored at a temperature of, with increasing preferences, less than 10 degrees Celsius, less than 0 degrees Celsius, less than −20 degrees Celsius wherein the composition comprises an MHC molecule having bound thereto in the peptide-binding groove of said MHC molecule a template peptide that dissociates from said MHC molecule at a temperature of 15 degrees Celsius or more, and preferably further comprises NaCl, preferably 100-600 mM NaCl, more preferably 250-350 mM NaCl and/or glycerol, preferably 1-50% (vol/vol) glycerol, preferably 5-15% (vol/vol) glycerol;
preferably wherein the MHC molecule is a multimer, wherein said MHC molecule is preferably a human HLA-A molecule, preferably a human HLA-A molecule selected from HLA-A*02 and HLA-A*02:01.

29-34. (canceled)

35. The composition of claim 28 wherein the template peptide is a polypeptide comprising
a. the polypeptide sequence as set forth in SEQ ID NO:1 (IAKEPVHGV), SEQ ID NO:2 (IAKEPVHGA) or SEQ ID NO:3 (FAPGNAPAL); or
b. the polypeptide sequence as set forth in SEQ ID NO:1 or SEQ ID NO:2 or SEQ ID NO:3 having 1, 2, 3, or 4 amino acid substitutions, deletions or insertions.
